(19) United States
(12) Patent Application Publication
(10) Pub. No.: US 2013/0244880 A1
(43) Pub. Date: Sep. 19, 2013

(54) Novel pesticide formulations

(71) Applicants: Michael William Burnet, Tubingen (DE); Jan-Hinrich Guse, Tubingen (DE); Martin Reisser, Esslingen (DE)

(72) Inventors: Michael William Burnet, Tubingen (DE); Jan-Hinrich Guse, Tubingen (DE); Martin Reisser, Esslingen (DE)

(73) Assignees: HiCap Formulations Ltd2, Nottingham (GB); Michael W. M. Burnet, Tuebingen (DE)

(21) Appl. No.: 13/385,916

(22) Filed: Mar. 14, 2012

(51) Int. Cl. A01N 25/10; A01P 13/00; B29B 9/00; A01P 3/00; A01P 7/04; A01P 5/00; A61K 9/00; A61K 9/14

(52) U.S. Cl. 504/360; 424/409; 424/484; 504/358; 424/486; 424/419; 264/12

(57) ABSTRACT

Bioactive substances are imbedded or mixed into functionalized matrices to form homogenous water-insoluble solid complexes with desirable field properties such as reduced leaching in soil, improved leaf retention (rainfastness), selective unloading to roots and convenient packaging and application. Bioactive substances that may be so complexed include pharmaceutical agents and pesticides including herbicides, insecticides, bacteriocides, rodenticides, nematicide and fungicides. The matrices comprise either a monomeric-, oligomeric- or (co)polymeric backbone which may be derivatized with chemical groups that exhibit ionic (amines, carboxyls), hydrophobic, and ligand-binding interactions to form the matrix of the formulation. The various matrices may be mixed with additives or modifiers, grafted, or fused to obtain optimal properties. The matrix/pesticide formulations may be applied as granules, as suspensions, emulsions in sprays, as foams, or as coats for seeds and fertilizers. Alternatively they may be melted and sprayed as concentrates. The formulations may be applied to foliage, soil, irrigation water, construction materials, seeding materials, grains, and buildings.

### FIELD OF THE INVENTION

This invention relates to formulations of biologically active agents and more specifically to formulation with a particular focus on optimizing the matrices needed for its application. It builds on integrating principles of agronomy, soil science, and polymer chemistry in addition to agrochemistry, plant protection, and plant physiology.

### BACKGROUND OF THE INVENTION

The challenge in agrochemistry or other large scale field applications of chemicals such as herbicides, bacteriocides, rodenticides, nematicide and fungicides (together defined as pesticides) is to find ways of achieving control of the target organism while limiting the amount of the xenobiotic substance that is loaded into and is free-moving in the ecosystem by leaching or by aerosol drift. The amount of such chemicals that is required is a function of their potency, the ability to place the compound selectively and their susceptibility to removal either via destruction in the environment (metabolism, photolysis, etc.) or loss (leaching, drift). Unfortunately, environmentally desirable properties such as facile biodegradation or other loss may result in a need for frequent re-application and thus an increase in the load on the environment. Although there has been dramatic progress in identifying more potent compounds for use in pest control there has been rather less success in controlling the application or placing of these chemicals in such a way as to limit losses and maximize efficacy.

Optimal placement has a critical role in the function of such compounds: herbicides used in annual crops should retain activity at or near the soil surface to ensure that germinating weeds are exposed to the compound. These same compounds should not enter the subsoil where they may be taken up by trees or other deep-rooted species resulting in off-target effects. This presents the agrochemist with a paradox in that the properties of many successful herbicides are also those that result in effects on non-target species.

Similarly, systemic insecticides or fungicides would ideally be applied at seeding in small quantities that would remain with and protect the crop plant throughout its life cycle, however, for reasons of persistence, stability and economy, it is not generally feasible with available formulations to apply amounts at seeding that can provide the long periods of control.

A common goal of formulation is to prevent aggregation of the active ingredient following dilution; another is to ensure that during mixing and packaging, the product remains uniform, flowable and non-accretive. Yet another goal of formulation is to govern droplet size such that small droplets will not drift off target. Formulation to enhance performance once applied to the target in the field is, however, less common (see US 2007/0149409).

Various examples of formulation for enhanced field performance are known in the art and include, amongst others, use of encapsulation, granulation, surfactants, stickers, control of droplet size and rheology, as well as humectants. Very rarely does a single compound perform more than one function: there are commercially available, separate sticking agents, separate humectants, separate compounds that control droplet size and again, separate slow release formulations. Most available slow release formulations are bulky; the ratio of formulant to agent on a dry weight basis is over 4 and often 10 to 50 times more.

A hitherto poorly explored area in pesticide chemistry, (meaning the fields of agronomy, soil science and polymer chemistry in addition to agrochemistry, plant protection and plant physiology) is, however, that of mixed-function substituted matrices that have the capacity to retain and/or reversibly trap active ingredients to form water-insoluble solid complexes. Such matrices can be selected to suit the properties and the applications of the associated compound and thus extend its range of uses consistent with the current need described above. Many can simultaneously perform many of the requirements of a formulant described above.

The problems of achieving season-long control provide another example of the benefits of using tuned matrix-formulations. Certain herbicides, for example, are anions or cations and thus highly water-soluble. This means that they may not be used in residual control applications because they are readily washed off leaves or leached into soil beyond the desired activity zone by rainfall. This problem is typically solved by either applying a larger amount of herbicide to compensate for losses (expensive and potentially toxic to a crop and environmentally hazardous), applying a mixture (difficult to find combinations that have the same spectrum and crop safety) or making analogs with greater stability or soil binding (expensive to register and non-availability may reduce early season control).

Although the usual rules and regulations of chemical use and registration limit flexibility of firms in launching new pesticides, they are far less restrictive on inert carriers that are a part of formulants insofar as they are known to be safe. Thus, it is possible to foresee a situation in which a user could lower the proportion of free pesticide (for initial or foliar effect) and alter the various exchangeable forms to their particular circumstances.

A similar approach can be taken with fungicides, especially those where residual systemic activity is required. The matrix-formulations of fungicides can be incorporated into seed dressings or applied in furrow during seeding. Here properties of tuned slow release can be used to ensure that the fungicide has a longer duration of availability with equal or less active fungicide. Similarly, use of a matrix that unloads its active ingredient in the presence of hydrogen ions will make the compound only selectively available in the soil, either in the immediate environment of the root, or of fungal hyphae.

Slow release formulations of fertilizers, pesticides (including herbicides, Schreiber et al., 1987, Gerstl et. al., 1998) and drugs (Anand et al., 2001) are common (see reviews, Lewis and Cowsar, 1977, Patwardhan and Das, 1983, Hussain, M., 1989), yet there are only few reports of applying such formulations to crop seeds (U.S. Pat. No. 6,096,686). The use of charged polymers/biopolymers, specifically those basic in nature, as formulants for some pesticides, specifically for acidic herbicides, is known but the effort is aimed towards producing a better herbicide salt to improve its activity (EP 0 360 181).

One such application is the protection of crops from parasites. Coating herbicide resistant seeds with a selective herbicide can prevent attachment of parasites for a limited period, however, much of the herbicide is lost through leaching allowing weeds and the parasites to attack late in the season (Kanampiu et al. 2002). There is, therefore, reason for slow release throughout the season to increase the period of protection and reduce impact on the crop itself.

There are distinct types of slow release formulations that are appropriate for molecules such as the herbicides imazapyr and pyrithiobac and other ALS-inhibitor herbicides that are slightly phytotoxic to maize, (Abayo at al., 1998), including:

1) Covalent binding to a matrix that is either biodegraded or where the covalent linkage is slowly hydrolyzed. Anionic pesticides such as 2,4-D have been esterified to starch cellulose, and dextrans by such technologies, (Diaz et al., 2001, Jagtap, et al., 1983, and Mehltretter et al., 1974). There are also the known hybrid molecules, which covalently link two or more pesticides (U.S. Pat. No. 3,914,230). Registration requirements are far more comprehensive with a new molecule formed when there is a covalent linkage than when there is an ionic or hydrophobic binding of parent pesticidal compound, which remains in the parent form in the formulant association and not as a new molecule as with covalent binding.
(2) Strong, non-covalent interactions with special matrices. Various slow release formulations of pharmaceutical preparations have been developed by such means specifically for pharmaceuticals, (Anand et al., 2001), and in one case, for pesticides (US 20070149409). U.S. Pat. No. 6,096,686 and U.S. patent application 20080145343 do disclose methods and details that one skilled in the art can appreciate being general methodologies in the field of this invention. In addition, concentration of herbicide solutions and other non-novel details are described in the articles by Kanampiu et al., 2001, 2002, 2003, 2009.)

The use of weak ionic interactions to bind herbicides to chemically modified montmorrilinite clays has been reported (Mishael 2002a,b). The release of bound material from the two types of formulation described above can be further modulated by micro-encapsulation technologies that further control the rate of release (Schreiber et al., 1987, Tefft and Friend, 1993). Another means to modify availability is the use of insoluble salts of agents (Gressel and Joel, 2000).

We have demonstrated that use of slow release on seed coats (PCT/US03/20966) is advantageous in protecting plants from parasites.

Most chemical agents deployed in the environment to control pests and parasites are required to act for a specific duration of time in order to allow for control of pest and parasite invading from non-treated areas, those emerging at later times, or those who are inactive at the time of application of the agent. To this end, the chemist should either create a highly stable molecule, or re-apply that molecule at regular interval. Both these solutions are undesirable: Stable molecules may accumulate in the environment, while re-application is expensive.

### SUMMARY OF THE INVENTION

The instant invention provides solution to the problems encountered by the known technology. Here we disclose broadly applicable matrices that allow the preparation of chemical agents in such a way that their availability to the environment may be controlled by a range of factors including particle size, melting point, degree of cross-linking and proportion of alkaline and acidic functions. Chemical agents are dissolved in the matrices and the resulting mixture may be formed, extruded, sprayed as a melt, ground, emulsified, or otherwise prepared for application. The matrix is biologically degradable, sparingly soluble in water, amenable to disagregation by organisms (notably roots of plants) and other sources of protons, and able to promote the retention of chemical agents against concentration gradients in water.

In one aspect the present invention provides for the use of a matrix explicitly functionalized depending on needs as formulation agents in preparations of pesticidal ingredients (herbicides, fungicides, insecticides, nematicides, acaricides and rodenticides as well as other chemicals used in the wider environment) and/or pharmaceutical agents.

In one embodiment, the matrices may be comprised of a monomeric, oligomeric or (co)polymeric backbone. In another embodiment, the polymeric backbones are of synthetic or semi-synthetic origin. In both cases, the polymers may vary widely in length (for example, 50, 100, 200, 2000, 10000, 20000 units to greater than 50000) and may be formed of a range of repeat structures and linking arrangements including but not limited to those we have described as a “polymeric backbone” and esters, amides, ethers, glycols, alkanes, thiols, sulfones, lignins, and sugars (e.g. substituted polymers of glucose, chitin or chitosan), their derivatives and co-polymers and mixed polymers. The polymers are, by economic necessity, generally derived from bulk commodities and include but are not limited to: substituted celluloses, dextrans, polyimines, oligo- and polypeptides, styrenes, vinyls, hydroxybutyrates, starches, fructans, carbonates, paraffin derived, and lignins.

In another embodiment, the monomeric or oligomeric backbones are defined as reaction products of either a fatty acid or carboxylic acid with primary or secondary amino functions of an amine, polyamine, and/or an amino alcohol compound.

In a further embodiment, the matrix formulation contains modifiers and or additives that assist the ease of the preparation of the end product and to obtain optimal controlled release and anti-leaching properties.

In another embodiment, the mixture of the pesticidal ingredients and the matrices are maintained in a solvent as a solution or suspension.

In another embodiment, the substituted matrices may be positively or negatively charged or with strong hydrophobic binding groups.

In another embodiment, the backbone may have side chains that are composed of but not limited to amines, variable length carbon chains, alcohols, aromatic groups, sulfides, sulfonates, carboxy acids, halogens, chelating functions, glycols and hydrophobic binding domains.

In another embodiment, the backbone may be further grafted at their termini to introduce additional functions different from those of the repeat unit.

In one embodiment, the bioactive substance forms hydrophobic interactions with the matrix. In another, the matrix has humectant properties; in another embodiment the matrix is an ion exchanger.

In a preferred embodiment, the exchanger is a high capacity anion exchanger and is composed of naturally occurring functions such as primary, secondary, tertiary and quaternary amines. These include but are not limited to substituted polymers containing imines, imidazoles, dimethylamines, diethylamines, betaines and guanidines.

In another embodiment, the exchanger is a high capacity cation exchanger and includes functions such as sulfides, sulfonates, sulphoxyethyls, phosphates, carboxyalkanes, and carboxyls.

It is clear to those skilled in the art that the functionality described above can be introduced onto a variety of backbones using standard reactions known in the art such as those described in US 2007/0149409 and materials referenced therein hereby incorporated by reference.

In another aspect, the matrix may be used as products applied in water as dispersible formulations co-administered with water. In another embodiment the same types of matrices may be incorporated into solid formulations for use in broadcast application, seed dressings or other point applications. The matrix may assist in improving the solubility or packaging of the active ingredient in the concentrated form or assist in the re-suspension of the dry form of a formulation, in water.

In another embodiment, the substituted matrices may be soluble or remain as solid carriers, as pellets or as water-dispersible, micronized small particles and may used alone or in combination with other ingredients such as lipids or fatty acids to form microemulsions or microspheres. In another aspect, the pesticidal ingredients of formulations may be loaded onto the matrices during manufacture. In another embodiment, they may be loaded by the end user. In another embodiment, the matrices may incorporate coding via size distribution that can be used, in addition to improved efficacy, to identify source of product and counterfeit products.

In another embodiment, the matrices can be attached to solid supports, or themselves form insoluble beads or small fibers. These beads or fibers may be derivatized as for other polymers. The beads may be selected for positive buoyancy in which case they are of potentially enhanced utility in the control of floating aquatic weeds in the case of herbicides, or of surface borne larvae or disease pathogens in the case of insecticides and fungicides, respectively. The beads/supports may also be negatively buoyant for use in paddy rice where preferential distribution of the active ingredient to the upper water or lower sediment layer may improve efficacy.

In another embodiment, the various matrix-based formulations may be used as seed coats for the control of pathogens and parasites including weeds.

In a preferred embodiment, the formulation comprising of a matrix, pesticide, and/or modifier may be melted at a specific temperature to render a liquid mixture which may be strategically sprayed in melted form to the target application, wherein the mixture solidifies at certain environmental temperature and forms a coating directly at the target point. This methodology offers a slow and controlled release of the pesticide thus ensuring long-term application scheme. The possible applications for this methodology include but are not limited to field application, wood and stone handling, textile treatment, and seed coating. In another embodiment, the various matrices may be incorporated into a kit for research use to assist chemical developers in finding optimal formulations for either a new active ingredient, or a new formulation for a specific condition or use.

### BRIEF DESCRIPTION OF DRAWINGS

FIG. 1 Effect of various formulations of dicamba applied to the cotyledons of sunflower seedlings and determined by subsequent growth as measured by internode extension.

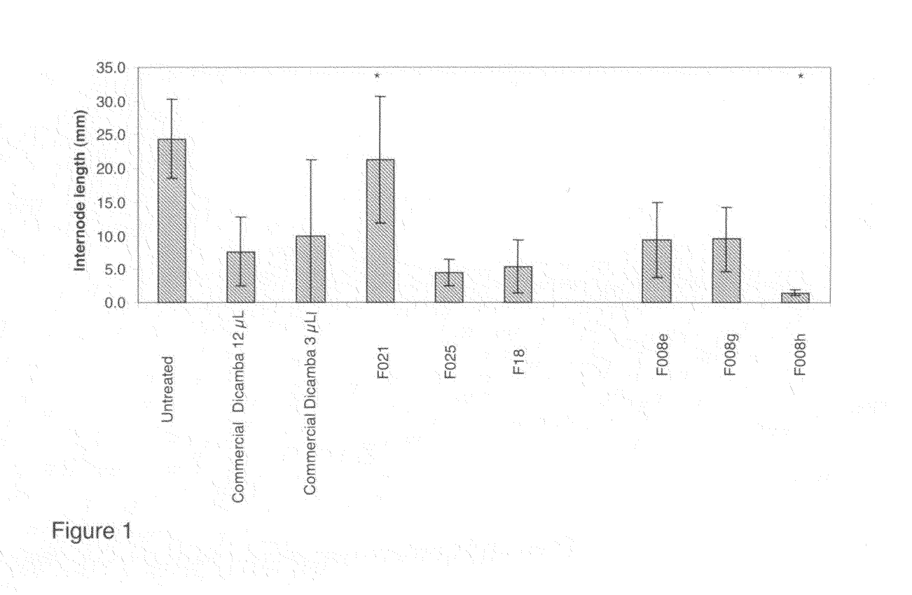

FIG. 2 Effect of various formulations of sulfentrazone applied to the cotyledons of sunflower seedlings and determined by subsequent growth as measured by internode extension.

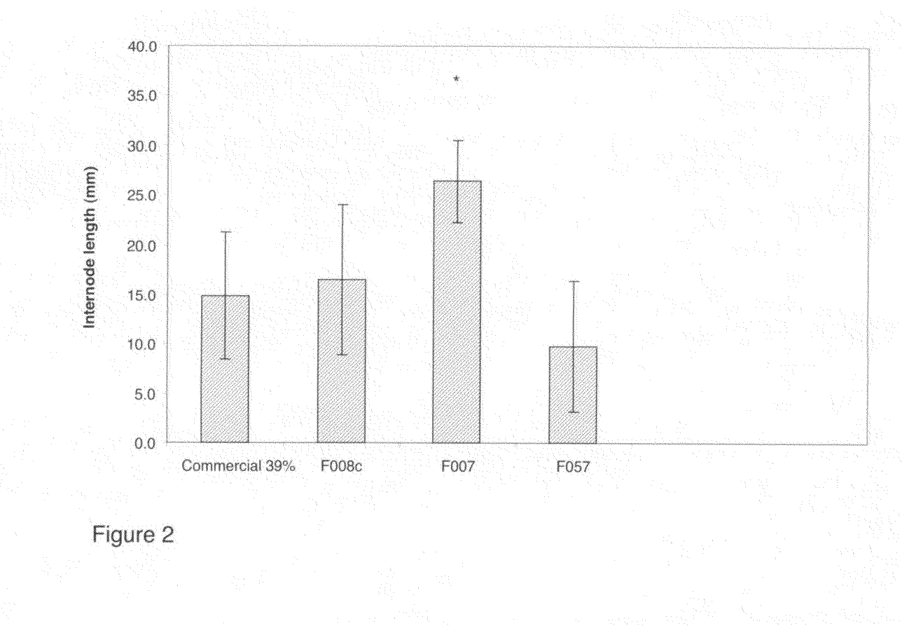

FIG. 3 Elution of sulfentrazone from soil columns when applied in two different formulations.

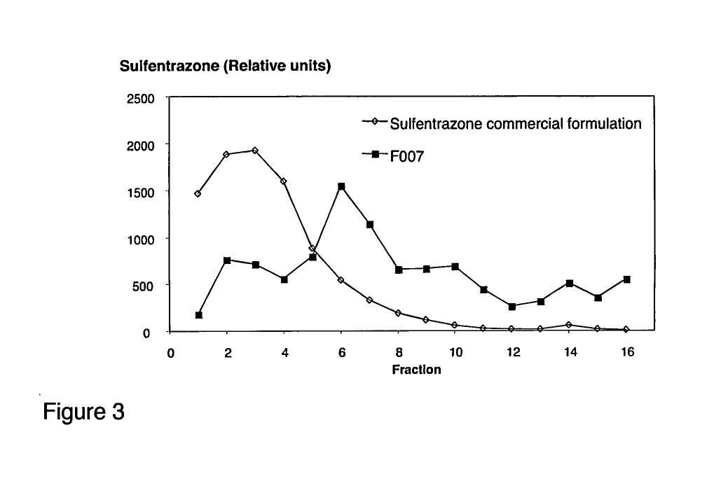

FIG. 4. Elution of mesotrione from soil columns when applied in three different formulations. Data are log concentration in relative units.

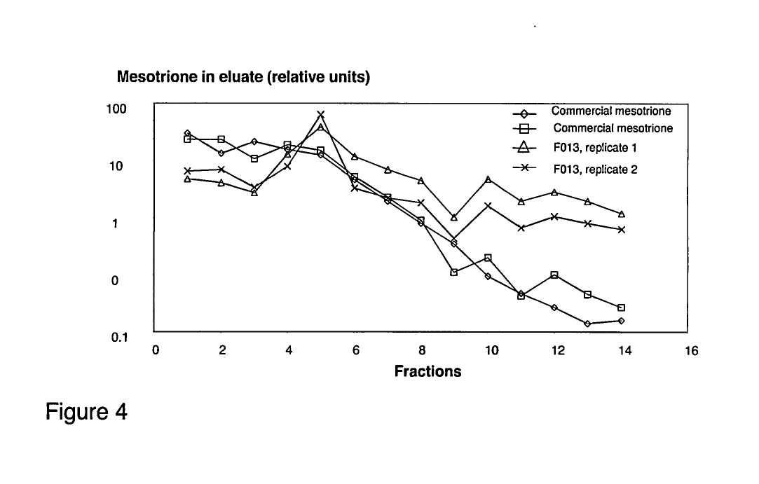

FIG. 5. Elution of terbuthylazine from soil columns when applied in three different formulations. Data are concentration in relative units.

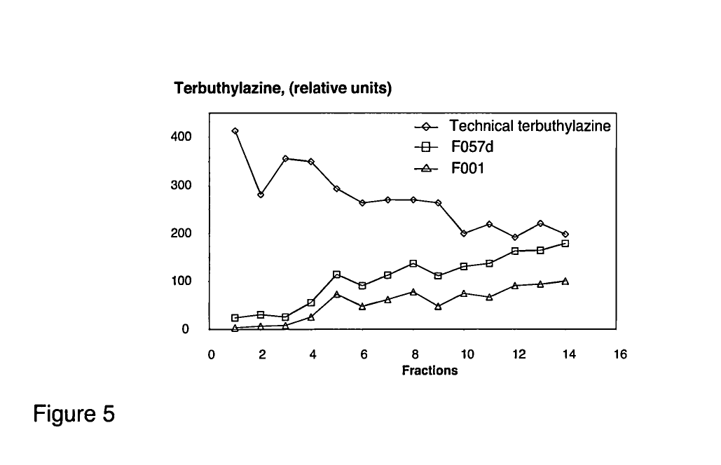

FIG. 6. Elution of 2,4-D from soil columns when applied in four different formulations. Data are concentration in relative units.

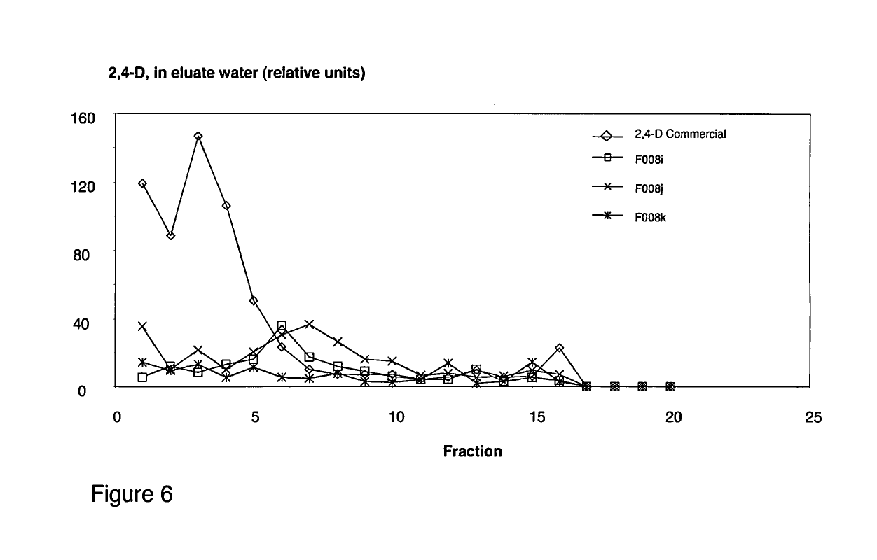

FIG. 7. Elution of quinclorac from soil columns when applied in four different formulations. Data are concentration in relative units.

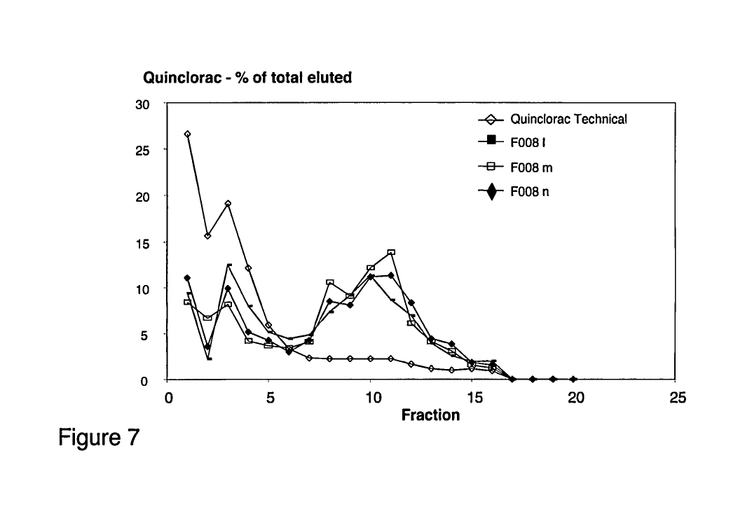

FIG. 8. Elution of sulfentrazone from soil columns when applied in a formulation ground to different size ranges. Data are concentration in ng/mL following application of the same amount of sulfentrazone.

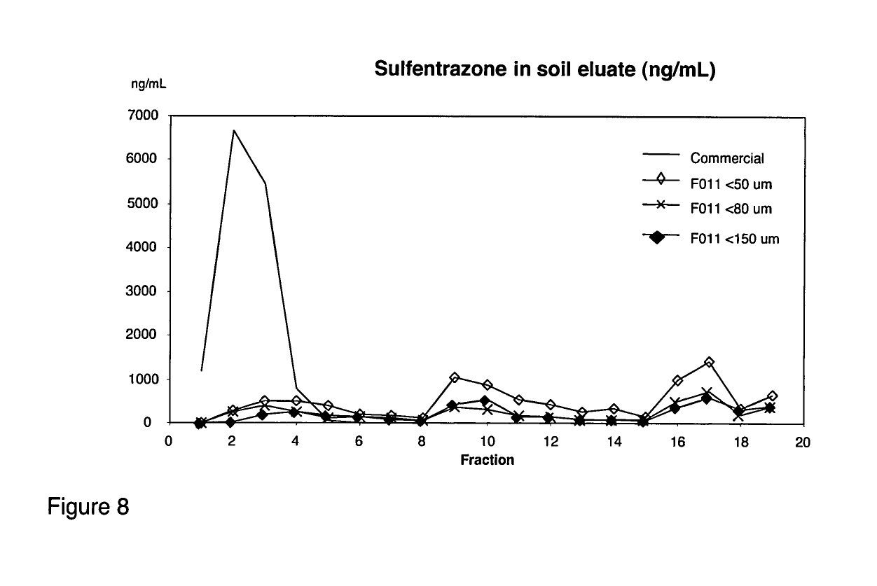

### DETAILED DESCRIPTION OF THE INVENTION

The general objective of formulation is to make a biologically active substance (bioactive substance) readily packagable as a concentrate, which may, in turn, be easily diluted and applied, typically via water. In pharmaceutical applications, a typical example is the use of excipients, surfactants and polymers to carry a hydrophobic substance into solution or emulsion for intravenous injection in an aqueous medium. In agrochemical applications, small molecules are also typically slightly hydrophobic in nature and not always readily soluble in spray solutions. Thus the goal of formulation is to allow intrinsically insoluble materials to be dispersed and applied via water vehicles.

In certain circumstances, it is desirable to provide a bioactive substance in such a way that it is available for a sustained period of time through a physical separation from the biological system. The material is, thereby protected from losses to the environment or from rapid elimination by a non-saturable process (pH mediated hydrolysis).

Several means of achieving this are well known in the art and include granulation and encapsulation. Granules are distributed by spreaders and generally have the disadvantage of low loading of active ingredient and correspondingly high costs of excipients and application. Encapsulation formulations are made by means of interfacial polymerization of emulsions containing the active ingredient. They are effective but are limited in the concentration of the species that they may carry, and are correspondingly expensive to produce.

In contrast, the present invention is an efficient means to formulate substances in which highly functionalized polymers comprise a matrix in which the substance is retained with high affinity and which in combination with properties of particle size, cross-linking and material stability, provide for a sustained release process leading to more stable concentrations of the formulated substance over time.

In a general sense, the matrices are made in a separate process (see examples 1-40) involving condensation and similar reactions that give rise to a solid that can be immediately mixed with the substance or substances to be formulated either as a molten material, or as a concentrate in a volatile solvent. In certain embodiments, the matrix may also be formed by mixing a free base of a fatty substance, and the free acid of a polymer, or oligomeric acid or divalent acid or fatty acid; or mixing a free organic acid with a the free base of a polymer, or oligomeric base or divalent base or aliphatic base.

Appropriately derivatized matrices are mixed with bioactive substances (i.e. pesticides) to form water-resistant solid complexes that exhibit desirable properties including sustained release, resistance to leaching through the soil, improved retention on leaf surfaces (rainfastness), selective unloading of compounds into the root environment and more convenient packaging and application. The matrices are generally bio-degradable, inexpensive, and regarded as safe with respect to toxicity. The matrices are composed mainly of a backbone derivatized with chemical groups that exhibit ionic interactions, hydrophobic interactions, complexing interactions (e.g. metal chelating) and ligand binding interactions. The matrix/bioactive substance mixtures may be mixed with additives or modifiers, grafted, or fused to obtain optimal controlled release and anti-leaching properties. The matrices exert their beneficial effects through binding to both the bioactive substance to be delivered and interaction with the leaf, soil or organic matter to modify pesticide exposure to the environment.

Specifically for the pesticides with which the invention may be useful include herbicides, insecticides, bacteriocides, rodenticides, nematicides and fungicides. Herbicides include but are not restricted to: imidazolinone herbicides, amitrole, glyphosate, glufosinate, carbetamide indole acetic acids, allidochlor, beflubutamid, benzadox, benzipram, bromobutide, cafenstrole, CDEA, chlorthiamid, cyprazole, dimethenamid, dimethenamid-P, diphenamid, epronaz, etnipromid, fentrazamide, flupoxam, fomesafen, halosafen, isocarbamid, isoxaben, napropamide, naptalam, pethoxamid, propyzamide, quinonamid, tebutam, anilide, herbicides, chloranocryl, cisanilide, clomeprop, cypromid, diflufenican, etobenzanid, fenasulam, flufenacet, flufenican, mefenacet, mefluidide, metamifop, monalide, naproanilide, pentanochlor, picolinafen, propanil, arylalanine, herbicides, benzoylprop, flamprop, flamprop-M, chloroacetanilide, herbicides, acetochlor, alachlor, butachlor, butenachlor, delachlor, diethatyl, dimethachlor, metazachlor, metolachlor, S-metolachlor, pretilachlor, propachlor, propisochlor, prynachlor, terbuchlor, thenylchlor, xylachlor, sulfonanilide, herbicides, benzofluor, cloransulam, diclosulam, florasulam, flumetsulam, metosulam, perfluidone, pyrimisulfan, profluazol, sulfonamide, herbicides, asulam, carbasulam, fenasulam, oryzalin, penoxsulam, see, also, sulfonylurea, herbicides, antibiotic, herbicides, bilanafos, sulfentrazone, aromatic, acid, herbicides, chloramben, dicamba, 2,3,6-TBA, tricamba, pyrimidinyloxybenzoic, acid, herbicides, bispyribac, pyriminobac, pyrimidinylthiobenzoic, acid, herbicides, pyrithiobac, phthalic, acid, herbicides, chlorthal, picolinic, acid, herbicides, aminopyralid, clopyralid, picloram, quinolinecarboxylic, acid, herbicides, quinclorac, quinmerac, arsenical herbicides, cacodylic acid, CMA, DSMA, hexaflurate, MAA, MAMA, MSMA, potassium arsenite, sodium arsenite, benzoylcyclohexanedione herbicides, mesotrione, sulcotrione, benzofuranyl, alkylsulfonate herbicides, benfuresate, ethofumesate, carbamate herbicides, asulam, carboxazole, chlorprocarb, dichlormate, fenasulam, karbutilate, terbucarb, carbanilate herbicides, barban, BCPC, carbasulam, carbetamide, CEPC, chlorbufam, chlorpropham, CPPC, desmedipham, phenisopham, phenmedipham, phenmedipham-ethyl, propham, swep, cyclohexene, oxime herbicides, alloxydim, butroxydim, clethodim, cloproxydim, cycloxydim, profoxydim, sethoxydim, tepraloxydim, tralkoxydim, cyclopropylisoxazole herbicides, isoxachlortole, isoxaflutole, dicarboximide herbicides, benzfendizone, cinidon-ethyl, flumezin, flumiclorac, flumioxazin, flumipropyn, dinitroaniline, herbicides, benfluralin, butralin, dinitramine, ethalfluralin, fluchloralin, isopropalin, methalpropalin, nitralin, oryzalin, pendimethalin, prodiamine, profluralin, trifluralin, dinitrophenol herbicides, dinofenate, dinoprop, dinosam, dinoseb, dinoterb, DNOC, etinofen, medinoterb, diphenyl ether herbicides, ethoxyfen, nitrophenyl ether herbicides, acifluorfen, aclonifen, bifenox, chlomethoxyfen, chlornitrofen, etnipromid, fluorodifen, fluoroglycofen, fluoronitrofen, fomesafen, furyloxyfen, halosafen, lactofen, nitrofen, nitrofluorfen, oxyfluorfen, dithiocarbamate herbicides, dazomet, metam, halogenated aliphatic herbicides, alorac, chloropon, dalapon, flupropanate, hexachloroacetone, iodomethane, methyl, bromide, monochloroacetic, acid, SMA, TCA, imidazolinone herbicides, imazamethabenz, imazamox, imazapic, imazapyr, imazaquin, imazethapyr, inorganic herbicides, ammonium sulfamate, borax, calcium chlorate, copper sulfate, ferrous sulfate, potassium azide, potassium cyanate, sodium azide, sodium chlorate, sulfuric acid, nitrile herbicides, bromobonil, bromoxynil, chloroxynil, dichlobenil, iodobonil, ioxynil, pyraclonil, organophosphorus herbicides, amiprofos-methyl, anilofos, bensulide, bilanafos, butamifos, 2,4-DEP, DMPA, EBEP, fosamine, glufosinate, glyphosate, piperophos, phenoxy, herbicides, bromofenoxim, clomeprop, 2,4-DEB, 2,4-DEP, difenopenten, disul, erbon, etnipromid, fenteracol, trifopsime, phenoxyacetic herbicides, 4-CPA, 2,4-D, 3,4-DA, MCPA, MCPA-thioethyl, 2,4,5-T, phenoxybutyric herbicides, 4-CPB, 2,4-DB, 3,4-DB, MCPB, 2,4,5-TB, phenoxypropionic herbicides, cloprop, 4-CPP, dichlorprop, dichlorprop-P, 3,4-DP, fenoprop, mecoprop, mecoprop-P, aryloxyphenoxypropionic, herbicides, chlorazifop, clodinafop, clofop, cyhalofop, diclofop, fenoxaprop, fenoxaprop-P, fenthiaprop, fluazifop, fluazifop-P, haloxyfop, haloxyfop-P, isoxapyrifop, metamifop, propaquizafop, quizalofop, quizalofop-P, trifop, phenylenediamine herbicides, dinitramine, prodiamine, phenyl pyrazolyl ketone herbicides, benzofenap, pyrazolynate, pyrazoxyfen, topramezone, pyrazolylphenyl herbicides, fluazolate, pyraflufen, pyridazine herbicides, credazine, pyridafol, pyridate, pyridazinone, herbicides, brompyrazon, chloridazon, dimidazon, flufenpyr, metflurazon, norflurazon, oxapyrazon, pydanon, pyridine herbicides, aminopyralid, cliodinate, clopyralid, dithiopyr, fluoroxypyr, haloxydine, picloram, picolinafen, pyriclor, thiazopyr, triclopyr, pyrimidinediamine herbicides, iprymidam, tioclorim, quaternary ammonium herbicides, cyperquat, diethamquat, difenzoquat, diquat, morfamquat, paraquat, thiocarbamate herbicides, butylate, cycloate, di-allate, EPTC, esprocarb, ethiolate, isopolinate, methiobencarb, molinate, orbencarb, pebulate, prosulfocarb, pyributicarb, sulfallate, thiobencarb, tiocarbazil, tri-allate, vernolate, thiocarbonate herbicides, dimexano, EXD, proxan, thiourea herbicides, methiuron, triazine herbicides, dipropetryn, triaziflam, trihydroxytriazine, chlorotriazine herbicides, atrazine, chlorazine, cyanazine, cyprazine, eglinazine, ipazine, mesoprazine, procyazine, proglinazine, propazine, sebuthylazine, simazine, terbuthylazine, trietazine, methoxytriazine herbicides, atraton, methometon, prometon, secbumeton, simeton, terbumeton, methylthiotriazine herbicides, ametryn, aziprotryne, cyanatryn, desmetryn, dimethametryn, methoprotryne, prometryn, simetryn, terbutryn, triazinone herbicides, ametridione, amibuzin, hexazinone, isomethiozin, metamitron, metribuzin, triazole herbicides, amitrole, cafenstrole, epronaz, flupoxam, triazolone herbicides, amicarbazone, carfentrazone, flucarbazone, propoxycarbazone, sulfentrazone, triazolopyrimidine, herbicides, cloransulam, diclosulam, florasulam, flumetsulam, metosulam, penoxsulam, uracil herbicides, butafenacil, bromacil, flupropacil, isocil, lenacil, terbacil, urea herbicides, benzthiazuron, cumyluron, cycluron, dichloralurea, diflufenzopyr, isonoruron, isouron, methabenzthiazuron, monisouron, noruron, phenylurea herbicides, anisuron, buturon, chlorbromuron, chloreturon, chlorotoluron, chloroxuron, daimuron, difenoxuron, dimefuron, diuron, fenuron, fluometuron, fluothiuron, isoproturon, linuron, methiuron, methyldymron, metobenzuron, metobromuron, metoxuron, monolinuron, monuron, neburon, parafluoron, phenobenzuron, siduron, tetrafluoron, thidiazuron, sulfonylurea herbicides, pyrimidinylsulfonylurea herbicides, amidosulfuron, azimsulfuron, bensulfuron, chlorimuron, cyclosulfamuron, ethoxysulfuron, flazasulfuron, flucetosulfuron, flupyrsulfuron, foramsulfuron, halosulfuron, imazosulfuron, mesosulfuron, nicosulfuron, orthosulfamuron, oxasulfuron, primisulfuron, pyrazosulfuron, rimsulfuron, sulfometuron, sulfosulfuron, trifloxysulfuron, triazinylsulfonylurea herbicides, chlorsulfuron, cinosulfuron, ethametsulfuron, iodosulfuron, metsulfuron, prosulfuron, thifensulfuron, triasulfuron, tribenuron, triflusulfuron, tritosulfuron, thiadiazolylurea herbicides, buthiuron, ethidimuron, tebuthiuron, thiazafluoron, thidiazuron, unclassified herbicides, acrolein, allyl, alcohol, azafenidin, benazolin, bentazone, benzobicyclon, buthidazole, calcium cyanamide, cambendichlor, chlorfenac, chlorfenprop, chlorflurazole, chlorflurenol, cinmethylin, clomazone, CPMF, cresol, ortho-dichlorobenzene, dimepiperate, endothal, fluoromidine, fluridone, fluorochloridone, flurtamone, fluthiacet, indanofan, methazole, methyl, isothiocyanate, nipyraclofen, OCH, oxadiargyl, oxadiazon, oxaziclomefone, pentachlorophenol, pentoxazone, phenylmercury acetate, pinoxaden, prosulfalin, pyribenzoxim, pyriftalid, quinoclamine, rhodethanil, sulglycapin, thidiazimin, tridiphane, trimeturon, tripropindan, tritac.

Fungicides include but are not limited to pyridine, carbamate and benzimidazole type fungicides (respectively cyprodinil, propamocarb, and carbendazim) penconazole, validamycin, kasugamycin, butylamine, azoxystrobin, aliphatic nitrogen fungicides, butylamine, cymoxanil, dodicin, dodine, guazatine, iminoctadine, amide fungicides, carpropamid, chloraniformethan, cyflufenamid, diclocymet, ethaboxam, fenoxanil, flumetover, furametpyr, mandipropamid, penthiopyrad, prochloraz, quinazamid, silthiofam, triforine, acylamino acid fungicides, benalaxyl, benalaxyl-M, furalaxyl, metalaxyl, metalaxyl-M, pefurazoate, anilide fungicides, benalaxyl, benalaxyl-M, boscalid, carboxin, fenhexamid, metalaxyl, metalaxyl-M, metsulfovax, ofurace, oxadixyl, oxycarboxin, pyracarbolid, thifluzamide, tiadinil, benzanilide fungicides, benodanil, flutolanil, mebenil, mepronil, salicylanilide, tecloftalam, furanilide fungicides, fenfuram, furalaxyl, furcarbanil, methfuroxam, sulfonanilide fungicides, flusulfamide, benzamide fungicides, benzohydroxamic acid, fluopicolide, tioxymid, trichlamide, zarilamid, zoxamide, furamide fungicides, cyclafuramid, furmecyclox, phenylsulfamide fungicides, dichlofluanid, tolylfluanid, sulfonamide fungicides, cyazofamid, valinamide fungicides, benthiavalicarb, iprovalicarb, antibiotic fungicides, aureofungin, blasticidin-S, cycloheximide, griseofulvin, kasugamycin, natamycin, polyoxins, polyoxorim, streptomycin, validamycin, strobilurin fungicides, azoxystrobin, dimoxystrobin, fluoxastrobin, kresoxim-methyl, metominostrobin, orysastrobin, picoxystrobin, pyraclostrobin, trifloxystrobin, aromatic fungicides, biphenyl, chlorodinitronaphthalene, chloroneb, chlorothalonil, cresol, dicloran, hexachlorobenzene, pentachlorophenol, quintozene, sodium pentachlorophenoxide, tecnazene, benzimidazole fungicides, benomyl, carbendazim, chlorfenazole, cypendazole, debacarb, fuberidazole, mecarbinzid, rabenzazole, thiabendazole, benzimidazole precursor fungicides, furophanate, thiophanate, thiophanate-methyl, benzothiazole fungicides, bentaluron, chlobenthiazone, TCMTB, bridged diphenyl fungicides, bithionol, dichlorophen, diphenylamine, carbamate fungicides, benthiavalicarb, furophanate, iprovalicarb, propamocarb, thiophanate, thiophanate-methyl, benzimidazolylcarbamate fungicides, benomyl, carbendazim, cypendazole, debacarb, mecarbinzid, carbanilate fungicides, diethofencarb, conazole fungicides, conazole fungicides (imidazoles), climbazole, clotrimazole, imazalil, oxpoconazole, prochloraz, triflumizole, see also imidazole fungicides, conazole fungicides (triazoles), azaconazole, bromuconazole, cyproconazole, diclobutrazol, difenoconazole, diniconazole, diniconazole-M, epoxiconazole, etaconazole, fenbuconazole, fluquinconazole, flusilazole, flutriafol, furconazole, furconazole-cis, hexaconazole, imibenconazole, ipconazole, metconazole, myclobutanil, penconazole, propiconazole, prothioconazole, quinconazole, simeconazole, tebuconazole, tetraconazole, triadimefon, triadimenol, triticonazole, uniconazole, uniconazole-P, see also triazole fungicides, copper fungicides, Bordeaux mixture, Burgundy mixture, Cheshunt mixture, copper acetate, basic copper carbonate, copper hydroxide, copper naphthenate, copper oleate, copper oxychloride, copper sulfate, basic copper sulfate, copper zinc chromate, cufraneb, cuprobam, cuprous oxide, mancopper, oxine copper, dicarboximide fungicides, famoxadone, fluoroimide, dichlorophenyl dicarboximide fungicides, chlozolinate, dichlozoline, iprodione, isovaledione, myclozolin, procymidone, vinclozolin, phthalimide fungicides, captafol, captan, ditalimfos, folpet, thiochlorfenphim, dinitrophenol fungicides, binapacryl, dinobuton, dinocap, dinocap-4,dinocap-6, dinocton, dinopenton, dinosulfon, dinoterbon, DNOC, dithiocarbamate fungicides, azithiram, carbamorph, cufraneb, cuprobam, disulfuram, ferbam, metam, nabam, tecoram, thiram, ziram, cyclic dithiocarbamate fungicides, dazomet, etem, milneb, polymeric dithiocarbamate fungicides, mancopper, mancozeb, maneb, metiram, polycarbamate, propineb, zineb, imidazole fungicides, cyazofamid, fenamidone, fenapanil, glyodin, iprodione, isovaledione, pefurazoate, triazoxide, see also conazole fungicides (imidazoles), inorganic fungicides, potassium azide, potassium thiocyanate, sodium azide, sulfur, see also copper fungicides, see also inorganic mercury fungicides, mercury fungicides, inorganic mercury fungicides, mercuric chloride, mercuric oxide, mercurous chloride, organomercury fungicides, (3-ethoxypropyl)mercury bromide, ethylmercury acetate, ethylmercury bromide, ethylmercury chloride, ethylmercury 2,3-dihydroxypropyl mercaptide, ethylmercury phosphate, N-(ethylmercury)-p-toluenesulphonanilide, hydrargaphen, 2-methoxyethylmercury chloride, methylmercury benzoate, methylmercury dicyandiamide, methylmercury pentachlorophenoxide, 8-phenylmercurioxyquinoline, phenylmercuriurea, phenylmercury acetate, phenylmercury chloride, phenylmercury derivative of pyrocatechol, phenylmercury nitrate, phenylmercury salicylate, thiomersal, tolylmercury acetate, morpholine fungicides, aldimorph, benzamorf, carbamorph, dimethomorph, dodemorph, fenpropimorph, flumorph, tridemorph, organophosphorus fungicides, ampropylfos, ditalimfos, edifenphos, fosetyl, hexylthiofos, iprobenfos, phosdiphen, pyrazophos, tolclofos-methyl, triamiphos, organotin fungicides, decafentin, fentin, tributyltin oxide, oxathiin fungicides, carboxin, oxycarboxin, oxazole fungicides, chlozolinate, dichlozoline, drazoxolon, famoxadone, hymexazol, metazoxolon, myclozolin, oxadixyl, vinclozolin, polysulfide fungicides, barium polysulfide, calcium polysulfide, potassium polysulfide, sodium polysulfide, pyrazole fungicides, furametpyr, penthiopyrad, pyridine fungicides, boscalid, buthiobate, dipyrithione, fluazinam, fluopicolide, pyridinitril, pyrifenox, pyroxychlor, pyroxyfur, pyrimidine fungicides, bupirimate, cyprodinil, diflumetorim, dimethirimol, ethirimol, fenarimol, ferimzone, mepanipyrim, nuarimol, pyrimethanil, triarimol, pyrrole fungicides, fenpiclonil, fludioxonil, fluoroimide, quinoline fungicides, ethoxyquin, halacrinate, 8-hydroxyquinoline sulfate, quinacetol, quinoxyfen, quinone fungicides, benquinox, chloranil, dichlone, dithianon, quinoxaline fungicides, chinomethionat, chlorquinox, thioquinox, thiazole fungicides, ethaboxam, etridiazole, metsulfovax, octhilinone, thiabendazole, thiadifluor, thifluzamide, thiocarbamate fungicides, methasulfocarb, prothiocarb, thiophene fungicides, ethaboxam, silthiofam, triazine fungicides, anilazine, triazole fungicides, bitertanol, fluotrimazole, triazbutil, see also conazole fungicides (triazoles), urea fungicides, bentaluron, pencycuron, quinazamid, unclassified fungicides, acibenzolar, acypetacs, allyl alcohol, benzalkonium chloride, benzamacril, bethoxazin, carvone, chloropicrin, DBCP, dehydroacetic acid, diclomezine, diethyl pyrocarbonate, fenaminosulf, fenitropan, fenpropidin, formaldehyde, furfural, hexachlorobutadiene, iodomethane, isoprothiolane, methyl bromide, methyl isothiocyanate, metrafenone, nitrostyrene, nitrothal-isopropyl, OCH, 2-phenylphenol, phthalide, piperalin, probenazole, proquinazid, pyroquilon, sodium orthophenylphenoxide, spiroxamine, sultropen, thicyofen, tricyclazole, zinc naphthenate, strobilurins such as azoxystrobin, picoxystrobin and others in this class.

Insecticides include thiocyclam, nicotine, CGA50439, cartap, allosamidin, thuringiensin, macrocyclic lactone insecticides, spinosad, avermectin insecticides, abamectin, doramectin, emamectin, eprinomectin, ivermectin, selamectin, milbemycin insecticides, lepimectin, milbemectin, milbemycin oxime, moxidectin, arsenical insecticides, calcium arsenate, copper acetoarsenite, copper arsenate, lead arsenate, potassium arsenite, sodium arsenite, botanical insecticides, anabasine, azadirachtin, d-limonene, nicotine, pyrethrins, cinerins, cinerin I, cinerin II, jasmolin I, acetamiprid, jasmolin II, pyrethrin I, pyrethrin II, quassia, rotenone, ryania, sabadilla, carbamate insecticides, bendiocarb, carbaryl, benzofuranyl methylcarbamate insecticides, benfuracarb, carbofuran, carbosulfan, decarbofuran, furathiocarb, dimethylcarbamate insecticides, dimetan, dimetilan, hyquincarb, pirimicarb, oxime carbamate insecticides, alanycarb, aldicarb, aldoxycarb, butocarboxim, butoxycarboxim, methomyl, nitrilacarb, oxamyl, tazimcarb, thiocarboxime, thiodicarb, thiofanox, phenyl methylcarbamate insecticides, allyxycarb, aminocarb, bufencarb, butacarb, carbanolate, cloethocarb, dicresyl, dioxacarb, EMPC, ethiofencarb, fenethacarb, fenobucarb, isoprocarb, methiocarb, metolcarb, mexacarbate, promacyl, promecarb, propoxur, trimethacarb, XMC, xylylcarb, dinitrophenol insecticides, dinex, dinoprop, dinosam, DNOC, fluorine insecticides, barium hexafluorosilicate, cryolite, sodium fluoride, sodium hexafluorosilicate, sulfluramid, formamidine insecticides, amitraz, chlordimeform, formetanate, formparanate, fumigant insecticides, acrylonitrile, imidadcloprid, carbon disulfide, carbon tetrachloride, chloroform, chloropicrin, para-dichlorobenzene, 1,2-dichloropropane, ethyl formate, ethylene dibromide, ethylene dichloride, ethylene oxide, hydrogen cyanide, iodomethane, methyl bromide, methylchloroform, methylene chloride, naphthalene, phosphine, sulfuryl fluoride, tetrachloroethane, inorganic insecticides, borax, calcium polysulfide, copper oleate, mercurous chloride, potassium thiocyanate, sodium thiocyanate, see also arsenical insecticides, see also fluorine insecticides, insect growth regulators, chitin synthesis inhibitors, bistrifluoron, buprofezin, chlorfluazuron, cyromazine, diflubenzuron, flucycloxuron, flufenoxuron, hexaflumuron, lufenuron, novaluron, noviflumuron, penfluoron, teflubenzuron, triflumuron, juvenile hormone mimics, epofenonane, fenoxycarb, hydroprene, kinoprene, methoprene, pyriproxyfen, triprene, juvenile hormones, juvenile hormone I, juvenile hormone II, juvenile hormone III, moulting hormone agonists, chromafenozide, halofenozide, methoxyfenozide, tebufenozide, moulting hormones, α-ecdysone, ecdysterone, moulting inhibitors, diofenolan, precocenes, precocene I, precocene II, precocene III, unclassified insect growth regulators, dicyclanil, nereistoxin analogue insecticides, bensultap, cartap, thiocyclam, thiosultap, nicotinoid insecticides, flonicamid, nitroguanidine insecticides, clothianidin, dinotefuran, imidacloprid, thiamethoxam, nitromethylene insecticides, nitenpyram, nithiazine, pyridylmethylamine insecticides, acetamiprid, imidacloprid, nitenpyram, thiacloprid, organochlorine insecticides, bromo-DDT, camphechlor, DDT, pp'-DDT, ethyl-DDD, HCH, gamma-HCH, lindane, methoxychlor, pentachlorophenol, TDE, cyclodiene insecticides, aldrin, bromocyclen, chlorbicyclen, chlordane, chlordecone, dieldrin, dilor, endosulfan, endrin, HEOD, heptachlor, HHDN, isobenzan, isodrin, kelevan, mirex, organophosphorus insecticides, organophosphate insecticides, bromfenvinfos, chlorfenvinphos, crotoxyphos, dichlorvos, dicrotophos, dimethylvinphos, fospirate, heptenophos, methocrotophos, mevinphos, monocrotophos, naled, naftalofos, phosphamidon, propaphos, TEPP, tetrachlorvinphos, organothiophosphate insecticides, dioxabenzofos, fosmethilan, phenthoate, aliphatic organothiophosphate insecticides, acethion, amiton, cadusafos, chlorethoxyfos, chlormephos, demephion, demephion-O, demephion-S, demeton, demeton-O, demeton-S, demeton-methyl, demeton-O-methyl, demeton-5-methyl, demeton-5-methylsulphon, disulfoton, ethion, ethoprophos, IPSP, isothioate, malathion, methacrifos, oxydemeton-methyl, oxydeprofos, oxydisulfoton, phorate, sulfotep, terbufos, thiometon, aliphatic amide organothiophosphate insecticides, amidithion, cyanthoate, dimethoate, ethoate-methyl, formothion, mecarbam, omethoate, prothoate, sophamide, vamidothion, oxime organothiophosphate insecticides, chlorphoxim, phoxim, phoxim-methyl, heterocyclic organothiophosphate insecticides, azamethiphos, coumaphos, coumithoate, dioxathion, endothion, menazon, morphothion, phosalone, pyraclofos, pyridaphenthion, quinothion, benzothiopyran organothiophosphate insecticides, dithicrofos, thicrofos, benzotriazine organothiophosphate insecticides, azinphos-ethyl, azinphos-methyl, isoindole organothiophosphate insecticides, dialifos, phosmet, isoxazole organothiophosphate insecticides, isoxathion, zolaprofos, pyrazolopyrimidine organothiophosphate insecticides, chlorprazophos, pyrazophos, pyridine organothiophosphate insecticides, chlorpyrifos, chlorpyrifos-methyl, pyrimidine organothiophosphate insecticides, butathiofos, diazinon, etrimfos, lirimfos, pirimiphos-ethyl, pirimiphos-methyl, primidophos, pyrimitate, tebupirimfos, quinoxaline organothiophosphate insecticides, quinalphos, quinalphos-methyl, thiadiazole organothiophosphate insecticides, athidathion, lythidathion, methidathion, prothidathion, triazole organothiophosphate insecticides, isazofos, triazophos, phenyl organothiophosphate insecticides, azothoate, bromophos, bromophos-ethyl, carbophenothion, chlorthiophos, cyanophos, cythioate, dicapthon, dichlofenthion, etaphos, famphur, fenchlorphos, fenitrothion, fensulfothion, fenthion, fenthion-ethyl, heterophos, jodfenphos, mesulfenfos, parathion, parathion-methyl, phenkapton, phosnichlor, profenofos, prothiofos, sulprofos, temephos, trichlormetaphos-3, trifenofos, phbsphonate insecticides, butonate, trichlorfon, phosphonothioate insecticides, mecarphon, phenyl ethylphosphonothioate insecticides, fonofos, trichloronat, phenyl phenylphosphonothioate insecticides, cyanofenphos, EPN, leptophos, phosphoramidate insecticides, crufomate, fenamiphos, fosthietan, mephosfolan, phosfolan, pirimetaphos, phosphoramidothioate insecticides, acephate, isocarbophos, isofenphos, methamidophos, propetamphos, phosphorodiamide insecticides, dimefox, mazidox, mipafox, schradan, oxadiazine insecticides, indoxacarb, phthalimide insecticides, dialifos, phosmet, tetramethrin, pyrazole insecticides, acetoprole, ethiprole, fipronil, pyrafluprole, pyriprole, tebufenpyrad, tolfenpyrad, vaniliprole, pyrethroid insecticides, pyrethroid ester insecticides, acrinathrin, allethrin, bioallethrin, barthrin, bifenthrin, bioethanomethrin, cyclethrin, cycloprothrin, cyfluthrin, beta-cyfluthrin, cyhalothrin, gamma-cyhalothrin, acetamiprid, lambda-cyhalothrin, cypermethrin, alpha-cypermethrin, beta-cypermethrin, theta-cypermethrin, zeta-cypermethrin, cyphenothrin, deltamethrin, dimefluthrin, dimethrin, empenthrin, fenfluthrin, fenpirithrin, fenpropathrin, fenvalerate, esfenvalerate, flucythrinate, fluvalinate, tau-fluvalinate, furethrin, imiprothrin, metofluthrin, permethrin, biopermethrin, transpermethrin, phenothrin, prallethrin, profluthrin, pyresmethrin, resmethrin, bioresmethrin, cismethrin, tefluthrin, terallethrin, tetramethrin, tralomethrin, transfluthrin, pyrethroid ether insecticides, etofenprox, flufenprox, halfenprox, protrifenbute, silafluofen, pyrimidinamine insecticides, flufenerim, pyrimidifen, pyrrole insecticides, chlorfenapyr, tetronic acid insecticides, spiromesifen, thiourea insecticides, diafenthiuron, urea insecticides, flucofuron, sulcofuron, see also chitin synthesis inhibitors, unclassified insecticides, closantel, crotamiton, EXD, fenazaflor, fenoxacrim, flubendiamide, hydramethylnon, isoprothiolane, malonoben, metaflumizone, metoxadiazone, nifluridide, pyridaben, pyridalyl, rafoxanide, triarathene, triazamate,

### Matrix

Matrices useful for the formulation and complexation of pesticides can be considered as twofold: the main backbone which may be primarily either a monomer, oligomer or (co)polymer and the specific functionalities attached to them based on the pesticide employed. The term “polymeric backbone” includes but is not limited to polyamides, polyimines, polyamines, glycols, vinyls, styrenes, polyacrylates examples of which may include: acrylonitrile butadiene styrene (ABS), polyamide (PA), polybutadiene, poly(butylene terephthalate) (PBT), polycarbonate, poly(ether sulphone) (PES, PES/PEES), poly(ether ether ketone)s (PEEK, PES/PEEK), polyethylene (PE), poly(ethylene glycol) (PEG), polyethyleneimine (PEI), poly(ethylene terephthalate) (PET), polyimide, polypropylene (PP), polystyrene (PS), styrene acrylonitrile (SAN), poly(trimethylene terephthalate) (PTT), polyurethane (PU), polyvinylchloride (PVC), polyvinyldifluorine (PVDF), poly(vinyl pyrrolidone) (PVP), Hydroxy-terminated polybutadiene, polymethyl methacrylate, polypyrrole, polyurea, polyurethane, polyvinyl acetate, rayon, nitrocellulose, nylon, phenol formaldehyde resin, polyacrylamide, polyacrylonitrile, polyaniline, polydiacetylenes, polyester as well as derivatives thereof.

Where the polymer itself does not interact strongly with a pesticide, it may be derivatized with functions that do make interaction possible. These derivatization processes are widely known in the art and are cited or described herein.

The choice of matrices having a monomeric or oligomeric backbone are defined as salts or reaction products of either an aliphatic amine and a carboxylate oligomer; or a fatty acid or carboxylic acid with a primary or secondary amino functions of an amine, polyamine, and/or an amino alcohol compound which include but are not limited to the following: N-Butyldiethanolamine, 2-(Butylamino)ethanol, N-(2-Hydroxyethyl)ethylene-diamine, Triethanolamine, Diethanolamine, 2-(Methylamino)ethanol, Diethylenetriamine, N-(2-Aminoethyl)-1,3-propane-diamine, 1,2-Bis(3-aminopropylamino)ethane, Bis(3-aminopropyl)-amine, 3,3′-Diamino-N-methyldipropylamine, Bis(hexamethylene)tri-amine, 1,4-Bis(3-aminopropyl)piperazine, N,N′-Bis(3-aminopropyl)-1,3-propanediamine, N,N′-Bis(2-aminoethyl)-1,3-propanedi-amine, Tetraethylenepentamine, Pentaethylene-hexamine. Matrices with a monomeric backbone may also be comprised of aliphatic amines with one to five amine or amide functions and a linear carbon chain of at least 12 carbon atomes. This includes amines derived from fatty acids, like for example tallowamine or products like Noram 42 or Dinoram 42 (® of Ceca).

Formulations of pesticides are made by forming a slurry or solution of the pesticide and matrix in an appropriate solvent followed by addition of the additive or modifier, where needed. Alternatively, the matrix and the pesticide can be melted together with or without the additive in such a way as to form a homogenous mixture. The procedure is performed, depending on the reagents, at room temperature or elevated temperature with vigorous stirring to assure homogeneity, and then by drying or concentration if the final product is not dry. Depending on one's requirement and condition the dry product may be ground at a temperature where it is brittle, or left ageing in a moist atmosphere and/or at elevated temperature to harden.

On the other hand the formulation blend comprising of a matrix, pesticide, and/or modifier may be melted at a specific temperature to render a liquid mixture which can be strategically applied/sprayed to the target objects, examples of which are, but not limited to field application, wood and stone handling, textile treatment, and seed coating. The liquid mixtures solidify at ambient temperature and forms a coating directly at the target point, or results in the formation of solid particles through cooling in ambient air that are then slow-release repositories of the substance. This methodology offers a slow and controlled release of the pesticide thus ensuring a long-term application scheme.

The matrices are prepared from common materials. To increase interaction with the pesticides, the backbones are derivatized to form functions that interact with the selected pesticides via hydrophobic, ionic, stacking or chelation means. Derivatizations include the addition of acid groups, basic groups, rings and alkyl chains. Derivatizations may be also used to cross-link polymers to form gels or more stable particles.

Formulations of pesticides are made by forming a slurry (or solution) of the pesticide and matrix in an appropriate solvent followed by addition of the additive or modifier, where needed. On the other hand, the matrix and the pesticide can be melted together with or without the additive in such a way as to form a homogenous mixture. The procedure is performed, depending on the reagents, at room temperature or elevated temperature with vigorous stirring to assure homogeneity, and then by drying or concentration if the final product is not dry. Depending on ones need the dry product may be ground with aid of liquid nitrogen, or left ageing in a moist atmosphere and/or at elevated temperature to harden. The invention will be further described in the following examples. It is clear to one skilled in the art that these examples are for illustrative purposes only and are not to be construed as limiting this invention in any manner.

### DEFINITIONS

Bioactive substance: A chemical composition that exerts an effect on an organism such as regulating growth, behaviour or homeostasis.

Pesticide or Pesticidal ingredient: A chemical composition that exerts a desirable effect on a pest species, said composition may, depending on its activity, be considered by those skilled in the art to be any of: herbicides, insecticides (including compounds controlling non-insect arthropods and nematodes), bacteriocides, rodenticides, and fungicides

Polymer: A chemical composition composed of repeating units covalently bound in a linear or networked (cross-linked) array. Polymers may be homo-(one repeating unit/monomers) or co-polymers (composed of multiple repeat units/monomers).

Monomer: A substance that possesses functionalities capable of being chemically bonded to itself or other monomers to form a polymer.

Oligomer: A chemical composition which consists of a limited number of monomer units.

Matrix: The substance which is often a major component in a formulation which may be defined as being either a monomeric, oligomeric- or (co)polymeric backbone which may be acidic or basic in nature, branched or linear, crosslinked or non-crosslinked and may exist in the free form or covalently linked to, mixed with and/or grafted with neutral components which include but are not limited to polyethylene glycol, propylene glycol, or fatty acid condensates, or a as reaction products of either a fatty acid or carboxylic acid with a primary or secondary amino functions of an amine, polyamine, and/or an amino alcohol compound. The matrix may be composed of a single substance or a mixture of substances. The matrix may be composed of alkyl acids or alkylamines alone or mixed.

Modifiers or Additives: A substance added during the formulation process to improve or modify the chemical, physical or biological properties of the end product, thus to obtain optimal controlled release and anti-leaching properties. Modifiers may be waxes, surfactants, ions, colourants, odours and neutral polymers.

Formulations of pesticides: Mixtures of a pesticidal ingredient and other chemical compositions with the effect of permitting preparation, storage and application of the ingredient.

Alkylamines are the same as fatty amines and the terms are used herein to mean primary, secondary or tertiary amines with at least one carbon chain longer than twelve units. Alkylacids are substances with an acidic function and at least one carbon chain longer than eight units.

Packaging: The means of containment by which formulations are weighed, stored, sold, transported, prepared for use and applied.

Beads and solid supports: Particles from 1 to 10000 microns that are water insoluble at pH 7.00 and which can directly, or following derivatization, interact with a pesticidal ingredient.

Rainfastness: The ability of a pesticidal ingredient to be held on a surface such that more than 5% of an amount deposited in a thin film on a leaf may be recovered from the leaf when said leaf is subject to simulated rainfall equivalent to 10 mm in an hour, said simulated rain commencing 120 minutes after application of the substance in conditions of relative humidity of 50% or less.

### ADVANTAGEOUS EFFECTS OF INVENTION

The invention described herein is a matrix system that may be easily adapted to incorporate a wide variety of chemical compounds and mediate their release to the environment over a sustained period. The Matrices have a very high capacity for compounds that allows practical formulations in which the compound may be a high proportion of the total weight of the preparation including ranges from 66% down to 5% or less.

The invention is now described by means of examples that are not meant to be limiting.

### Example 1

### Formulation to Retard Non-Polar Pesticide for a Longer Activity (F001)

a) 50 mL polyethylenimine solution (10% in methanol, containing 10% of water) and stearic acid (6.6 g) were combined and heated to 160° C. under a slow stream of argon under occasional system evacuation (ca. 150 mbar) and then vacume to 75 min, then vacuum is released, temperature, increased to 177° C. for an additional 30 min.
b) 400 mg of the product a) and terbutylazine (400 mg) were combined in a mortar and mixed by intense grinding at elevated temperature (50° C.). The pesticide mixture was cooled via addition of liquid nitrogen, and the material was pulverized to a fine powder.

### Example 2

### Preparation of a Slow Release Formulation of Sulfentrazone to a Solid Cationic Exchange Resin Based on Polyethylenimine (F002)

a) Jeffamine ED 900 (Huntsman, 13.72 g) and epichlorohydrin (7.9 mL) were dissolved in sufficient amounts of acetonitrile to give a total volume of 60 mL. The mixture was heated to reflux for 4 h.
b) Polyethylenimine (50% in H₂O=6.64 g of PEI, 13.27 g), 35 mL of solution a) and 35 mL of 0.1M NaOH were heated to 80° C. and to this mixture was added sufficient water to achieve a homogenous solution. Heating was continued for 24 h and the resulting gel was crushed with a mixing tool to a particle size of approx. 100 μm or smaller. When necessary, the mixture was kept fluid with the addition of water, at which point 3 mL of ethanolamine was added, and the mixture was heated to reflux for 3 h. The solids were isolated by centrifugation, and the material was washed 4 times with 12 time its volume with methanol. The dry weight of the material can be estimated after air drying and lies typically between 3 and 10%.
c) 1.7 g of product b) (10% dry mass=170 mg), and sulfentrazone (26% total, 60 mg) were combined and 3 mL of methanol were added. The mixture was shaken for 14 h and air-dried. The material is pulverized in a mortar with the aid of liquid nitrogen to yield a white, sticky powder.

The same procedure can be applied to other active ingredient. Examples of which are, but not limited to bromacil, quinclorac, 2,4-D, or dicamba. In addition, fungicides, insecticides, nematicides and other pest controlling agents may be treated the same way.

### Example 3

### Preparation of Slow Release Formulations with a Solid Ion Exchange Resin Based on Polyethylenimine and Silica (F003)

Dicamba (100 mg) was dissolved in 1 mL of a solution of 10% polyethylenimine and 10% water in methanol. Dissolution was sluggish and was accelerated by gentle warming and vigorous agitation. Tetraethyl orthosilicate (TEOS) (385 μL of a 13% solution) in ethanol were added, and the mixture was homogenized by shaking or stirring. After 45 min in a closed vessel, the mixture was transferred into an open container and left to air-dry for 12 h. The flexible, solid product can be milled when cooled i.e. with liquid nitrogen, or left for ageing in a moist atmosphere and/or at elevated temperature to harden.

The same procedure can be applied to other active ingredient. Examples of which are, but not limited to bromacil, sulfentrazone, quinclorac, or imazamox. In addition, fungicides, insecticides, nematicides and other pest controlling agents can be treated the same way.

### Example 4

### Preparation of a Non-Crystalline Formulation with High Load of Active Ingredient Specifically Herbicides (F004)

Sulfentrazone (100 mg) were dissolved in 430 μL of 10% polyethylenimine (PEI)/10% water in methanol (43 mg of PEI). Dissolution was sluggish, which was accelerated by gentle warming and vigorous agitation. TEOS solution (2.1M in abs. ethanol, previously treated with 0.035M acetyl chloride and pre-hydrolyzed with 0.5 equivalents of water, 165 μL) and SiO₂ (21.5 mg) were added. The mixture was shaken until homogeneity was achieved. After standing for 45 min in a closed vessel, the mixture is transferred into an open bin and left drying for 12 h.

The flexible solid product can be milled when cooled i.e. with liquid nitrogen, or left for ageing in a moist atmosphere or/and at elevated temperature to harden.

The same procedure can be applied to other active ingredient. Examples of which are, but not limited to bromacil, dicamba, quinclorac, or imazamox. In addition, fungicides, insecticides, nematicides and other pest controlling agents can be treated the same way.

### Example 5

### Preparation of a Mesotrione Iron Complex (F005)

Iron-(III)-chloride (1.6 g) was dissolved in 140 mL of dry THF. Mesotrione (9.33 g) was added and the mixture was shaken to produce a homogenous solution. Powdered iron (1.5 g) were added and the mixture was agitated in a closed vessel for 5 days. The precipitate was filtered off, washed with THF and ether, air-dried, and ground to a fine powder. Residual elemental iron was removed with the aid of a magnet. The product was obtained as a brown powder (5 g).

Alternate procedure: Iron-(III)-chloride (2 g) in 40 mL of dry THF were vigorously agitated with iron powder (1.6 g) overnight with the exclusion of air. Mesotrione (10 g) was added as a powder, and the mixture was shaken extensively. The precipitates were filtered off and washed with warm THF. The product crystallized out from the filtrate.

### Example 6

### Preparation of Slow Release Formulations with a Solid Cationic Exchange Resin Based on poly(diallyldimethylammonium chloride) and Silica (F006)

Glyphosate (100 mg, dissolved in 1 mL of dilute ammonia), 500 μL of a 20% solution of poly(diallyldimethylammonium chloride) in water, and 385 μL of a 2.1M solution of TEOS in ethanol were mixed and left standing in a closed vessel for 45 min until a clear viscosity increase could be seen. The mixture was air dried in an open vessel for 12 h. The product was pulverized with the aid of liquid nitrogen or left ageing in a moist atmosphere or/and at elevated temperature.

### Example 7

### Preparation of a Slow Release Formulation of Sulfentrazone with a Fatty Amine (F007)

Stearylamine (500 mg) and sulfentrazone (500 mg) were dissolved in approximately 10 mL of ethanol by slight heating. To this mixture was added 180 μL of a 20% solution of polyacrylic acid in ethanol (ca. 36 mg). A flocculent precipitation occurs. The reaction mixture is air-dried in an open vessel and left to harden for 4 to 7 days. The resulting slightly brittle white material can be carefully milled to a particle size of less then 50 μm and then be applied as such.

### Example 8

### Preparation of a Slow Release Formulation of Various Herbicides to a Solid Cationic Exchange Resin Based on Polyethylenimine (F008)

a) Jeffamine ED 900 (Huntsman, 50 g) and epichlorohydrin (30 mL) were dissolved in acetonitrile to give a total volume of 200 mL. The mixture was heated to reflux for 4 h, and acetonitrile was added to give a total volume of 250 mL.
b) Polyethylenimine (50% in H₂O=12.85 g of PEI, 25.7 g), and 32.5 mL of solution a) were dissolved in approximately 200 mL of methanol and heated to reflux overnight. The resulting gel was crushed with a mixing tool to a particle size of approximately 100 μm or smaller. When necessary, the mixture was kept fluid by addition of water. 1 mL of ethanolamine was added, and the mixture was heated to reflux for 30 min. All solids were isolated by centrifugation, and the material was washed with 1M aqueous potassium hydroxide solution, until no chloride can be detected in the washings with silver nitrate in dilute nitric acid. The gel was washed with water until the washings had a pH below 8.5, and then twice with 10 volumes of methanol. The material is air-dried and formed pale ochre agglutinating granules.
c) A sample of product b) was combined with a sample of active ingredient, enough methanol was added to keep the mixture fluid (it is not necessary to dissolve the active ingredient) and the mixture was shaken for 12 h. After air drying, a product remains which can be directly used. The different formulations are shown below:

TABLE 1
Different Formulations for Example 8

| | Active Ingredient | Amount (AI) | Amount Matrix b) | % AI |
|---|---|---|---|---|
| Entry | (AI) | [mg] | [mg] | (w/w) |
| F008a | sulfentrazone | 70 | 156 | 31 |
| F008b | sulfentrazone | 2940 | 5400 | 35 |
| F008c | sulfentrazone | 156 | 192 | 45 |
| F008d | sulfentrazone | 238 | 159 | 60 |
| F008e | dicamba | 78 | 183 | 30 |
| F008f | dicamba | 2700 | 4000 | 40 |
| F008g | dicamba | 123 | 150 | 45 |
| F008h | dicamba | 126 | 84 | 60 |
| F008i | 2,4-D | 93 | 216 | 30 |
| F008j | 2,4-D | 2530 | 3130 | 45 |
| F008k | 2,4-D | 127 | 84 | 60 |
| F008l | quinclorac | 88 | 195 | 31 |
| F008m | quinclorac | 91 | 111 | 45 |
| F008n | Quinclorac | 138 | 91 | 60 |

### Example 9

### Preparation of a Slow Release Formulation of Clomazone with a Urethane Matrix (F009)

Clomazone (167 mg) and matrix (example 29, M019, 1.4 g) were dissolved in ethanol and concentrated by evaporation. After vacuum-drying (ca. 2 h at 3 mbar), a white rubbery foam remains, that was pulverized with the aid of liquid nitrogen. Quantification of the active ingredient showed 10.8% content.

### Example 10

### Preparation of a Slow Release Formulation of Metribuzin with Colophony in an Acidic Matrix (F010)

Metribuzin (45 mg) were dissolved in 360 μL of a 25% solution of colophony in ethanol. To the resulting solution were added triethoxy(octadecyl)silane (35 mg), and 530 μL of a 2.1M solution of tetraethoxysilane in ethanol. After a homogenous mixture formed, the mixture was air-dried overnight. The resulting product was exposed to water (ca. 4 mL), and left to dry once again. The product, which was brittle and hard, was pulverized to a particle size of 100 μm or smaller. Analysis with HPLC/UV gives an active ingredient content of 16%.

### Example 11

### Preparation of a Matrix (M001)

Pentaethylenehexamine (23.2 g, 0.1 mol) and stearic acid (56.8 g, 0.2 mol) were mixed together. Sodium hypophosphite (0.1 g) was added. The mixture was heated to 135° C. for 1 h and at 175° C. for an additional 5 h until water formation ceased. The resulting product was a waxy, slightly yellow substance.

### Example 12

### Preparation of a Matrix (M002)

Pentaethylenehexamine (23.2 g, 0.1 mol), stearic acid (28.4 g, 0.1 mol) and oleic acid (28.2 g, 0.1 mol) were mixed together. Sodium hypophosphite (0.1 g) was added. The mixture was heated to 135° C. for 1 h and at 175° C. for an additional 5 h until water formation ceased. The resulting product was a pale yellow paste.

### Example 13

### Preparation of a Matrix (M003)

Pentaethylenehexamine (23.2 g, 0.1 mol) and stearic acid (56.8 g, 0.2 mol) were mixed together. Sodium hypophosphite (0.1 g) was added. The mixture was heated to 135° C. for 1 h and at 175° C. for an additional 5 h until water formation ceased. Then adipic acid (7.3 g, 0.05 mol) was added. The mixture was heated to 175° C. for an additional 5 h at reduced pressure (0.1 mbar). The resulting product was a waxy, light brown substance.

### Example 14

### Preparation of a Matrix (M004)

Pentaethylenehexamine (23.2 g, 0.1 mol), stearic acid (28.4 g, 0.1 mol) and oleic acid (28.2 g, 0.1 mol) were mixed together. Sodium hypophosphite (0.1 g) was added. The mixture was heated to 135° C. for 1 h and at 175° C. for an additional 5 h until water formation ceased. Then sebacic acid (10.1 g, 0.05 mol) was added. The mixture was heated to 175° C. for an additional 5 h at reduced pressure (0.1 mbar). The resulting product was a light brown paste.

### Example 15

### Preparation of a Matrix (M005)

Tetraethylenepentamine (18.9 g, 0.1 mol), stearic acid (28.4 g, 0.1 mol) and oleic acid (28.2 g, 0.1 mol) were mixed together. Sodium hypophosphite (0.1 g) was added. The mixture was heated to 135° C. for 1 h and at 175° C. for an additional 5 h until water formation ceased. Then sebacic acid (10.1 g, 0.05 mol) was added. The mixture was heated to 175° C. for an additional 5 h at reduced pressure (0.1 mbar). The resulting product was a light brown paste.

### Example 16

### Preparation of a Matrix (M006)

Pentaethylenehexamine (23.2 g, 0.1 mol), tetradecanoic acid (45.6 g, 0.2 mol) and oleic acid (56.4 g, 0.2 mol) were mixed together. Sodium hypophosphite (0.1 g) was added. The mixture was heated to 135° C. for 1 h and at 175° C. for an additional 5 h until water formation ceased. The resulting product was a light brown paste.

### Example 17

### Preparation of a Matrix (M007)

Triethylenetetramine 60% (24 g, 0.1 mol), 2-ethylhexanoic acid (14.4 g, 0.1 mol) and tetradecanoic acid (22.8 g, 0.1 mol) were mixed together. Sodium hypophosphite (0.1 g) was added. The mixture was heated to 135° C. for 1 h and at 175° C. for an additional 5 h until water formation ceased. The resulting product was a brown oil.

### Example 18

### Preparation of a Matrix (M008)

Tetraethylenepentamine (37.8 g, 0.2 mol) and stearic acid (113.6 g, 0.4 mol) were mixed together. Sodium hypophosphite (0.5 g) was added. The mixture was heated to 135° C. for 1 h and at 175° C. for an additional 5 h until water formation ceased. Then sebacic acid (10.1 g, 0.05 mol) was added. The mixture was heated to 175° C. for an additional 5 h at reduced pressure (0.1 mbar). The resulting product was a waxy, yellow solid.

Nitrogen number: 3.57 mmoles/g Tertiary nitrogen: 2.69 mmoles/g

### Example 19

### Preparation of a Matrix (M009)

Triethylenetetramine 60% (24 g, 0.1 mol) and stearic acid (56.8 g, 0.2 mol). Sodium hypophosphite (0.2 g) was added. The mixture was heated to 135° C. for 1 h and at 175° C. for an additional 5 h until water formation ceased. The resulting product was a waxy, yellow solid. Nitrogen number: 3.79 mmoles/g

### Example 20

### Preparation of a Matrix (M010)

N-(2-Hydroxyethyl)ethylendiamine (20.8 g, 0.2 mol) and stearic acid (113.6 g, 0.4 mol) were mixed together. Sodium hypophosphite (0.5 g) was added. The mixture was heated to 135° C. for 1 h and at 175° C. for an additional 5 h until water formation ceased. The mixture was heated to 175° C. for an additional 5 h at reduced pressure (0.1 mbar). The resulting product was a waxy, light brown solid.

Nitrogen number: 0.74 mmoles/g

### Example 21

### Preparation of a Matrix (M011)

N-(2-Hydroxyethyl)ethylendiamine (10.4 g, 0.1 mol), stearic acid (28.4 g, 0.1 mol) and oleic acid (28.2 g, 0.1 mol) were mixed together. Sodium hypophosphite (0.5 g) was added. The mixture was heated to 135° C. for 1 h and at 175° C. for an additional 5 h until water formation ceased. The mixture was heated to 175° C. for an additional 5 h at reduced pressure (0.1 mbar). The resulting product was a light brown paste.

### Example 22

### Preparation of a Matrix (M012)

N-(2-Hydroxyethyl)ethylendiamine (20.8 g, 0.2 mol) and stearic acid (101.4 g, 0.357 mol) were mixed together. Sodium hypophosphite (0.5 g) was added. The mixture was heated to 135° C. for 1 h and at 175° C. for an additional 5 h until water formation ceased. The mixture was heated to 175° C. for an additional 5 h at reduced pressure (0.1 mbar). The resulting product was a waxy, light brown solid.

### Example 23

### Preparation of a Matrix (M013)

Tetraethylenepentamine (37.8 g, 0.2 mol), stearic acid (56.8 g, 0.2 mol) and oleic acid (56.4 g, 0.2 mol) were mixed together. Sodium hypophosphite (0.5 g) was added. The mixture was heated to 135° C. for 1 h and at 175° C. for an additional 5 h until water formation ceased. The mixture was heated to 175° C. for an additional 5 h at reduced pressure (0.1 mbar). The resulting product was a light yellow paste.

Nitrogen number: 3.70 mmoles/g Tertiary Nitrogen: 3.38 mmoles/g

### Example 24

### Preparation of a Matrix (M014)

1,4-Bis(3-aminopropyl)piperazine (50 g, 0.25 mol) and stearic acid (142 g, 0.5 mol) were mixed together. Sodium hypophosphite (0.1 g) was added. The mixture was heated to 135° C. for 1 h and at 175° C. for an additional 5 h until water formation ceased. The mixture was heated to 175° C. for an additional 5 h at reduced pressure (0.1 mbar). The resulting product was a waxy, nearly colorless solid. Nitrogen number: 2.74 mmoles/g

### Example 25

### Preparation of a Matrix (M015)

1,4-Bis(3-aminopropyl)piperazine (40.06 g, 0.2 mol), stearic acid (56.8 g, 0.2 mol) and oleic acid (56.4 g, 0.2 mol) were mixed together. Sodium hypophosphite (0.5 g) was added. The mixture was heated to 135° C. for 1 h and at 175° C. for an additional 5 h until water formation ceased. The mixture was heated to 175° C. for an additional 5 h at reduced pressure (0.1 mbar). The resulting product was a waxy, light yellow solid.

Nitrogen number: 2.74 mmoles/g

### Example 26

### Preparation of a Matrix (M016)

1,4-Bis(3-aminopropyl)piperazine (40.06 g, 0.2 mol) and oleic acid (112.8 g, 0.4 mol) were mixed together. Sodium hypophosphite (0.5 g) was added. The mixture was heated to 135° C. for 1 h and at 175° C. for an additional 5 h until water formation ceased. The mixture was heated to 175° C. for an additional 8 h at reduced pressure (0.1 mbar). The resulting product was a waxy, yellow solid. Nitrogen number: 2.69 mmoles/g

### Example 27

### Preparation of a Matrix (M017)

Tetraethylenepentamine (37.8 g, 0.2 mol) and stearic acid (113.6 g, 0.4 mol) were mixed together. Sodium hypophosphite (0.5 g) was added. The mixture was heated to 135° C. for 1 h and at 175° C. for an additional 5 h until water formation ceased. The mixture was heated to 175° C. for an additional 5 h at reduced pressure (0.1 mbar). Then sebacic acid (10.1 g, 0.05 mol) was added. The mixture was heated to 175° C. for an additional 5 h at reduced pressure (0.1 mbar). The resulting product was a waxy, yellow solid.

Nitrogen number: 2.8 mmoles/g Tertiary Nitrogen: 2.61 mmoles/g

### Example 28

### Preparation of a Matrix (M018)

Condensate (50 g) from example 18 (M008) was melted and mixed with 4-Chlorophenyl-isocyanate (10.9 g, 71 mmol). The mixture was kept molten for 15 min. The product obtained was a Waxy, light yellow solid. Nitrogen number: 2.0 mmoles/g

### Example 29

### Preparation of a Matrix (M019)

Condensate (50 g) from example 18 (M008) was melted and mixed with 4-Chlorophenyl-isocyanate (22 g, 143 mmoles). The mixture is kept molten for 15 min. Slightly brittle, light yellow solid. Nitrogen number: 1.0 mmoles/g

### Example 30

### Preparation of a Matrix (M020)

Matrix (15.24 g) from example 12 (M002) was melted at 50° C. and mixed with Hexamethylene diisocyanate (1.68 g). The mixture was kept at 50° C. for another 30 min. The resulting product was a honey-like yellow paste.

### Example 31

### Preparation of a Matrix (M021)

Matrix (16.9 g) from example 14 (M004) was melted at 50° C. and mixed with Hexamethylene diisocyanate (1.68 g). The mixture was kept at 50° C. for another 30 min. The resulting product was a resin-like yellow paste.

### Example 32

### Preparation of a Matrix (M022)

Matrix (14.4 g) from example 18 (M008) was melted at 50° C. and mixed with Hexamethylene diisocyanate (1.68 g). The mixture was kept at 50° C. for another 30 min. The resulting product was a waxy, yellow solid.

### Example 33

### Preparation of a Matrix (M023)

Based on example 18 (M008), tetraethylenepentamine (37.8 g, 0.2 mol) and stearic acid (113.6 g, 0.4 mol) were mixed together. Sodium hypophosphite (0.5 g) was added. The mixture was heated to 135° C. for 1 h and at 175° C. for an additional 5 h until water formation ceased. The mixture was heated to 175° C. for an additional 8 h at reduced pressure (0.1 mbar). Nitrogen number: 3.72 mmoles/g Tertiary nitrogen: 2.87 mmoles/g The liquid mixture was then acetylated using acetic anhydride (12 g, 0.117 mol) and kept molten for 30 min. Nitrogen number: 2.69 mmoles/g

### Example 34

### Preparation of a Matrix (M024)

Matrix (15 g) from example 19 (M009) was dissolved in boiling isopropanol (approximately 40 mL). To this solution methyl iodide (6.63 g, 46.7 mmol) was added dropwise. Stirring was continued for 30 min, at which point, the solvent was evaporated in vacuo. The resulting product was a yellow solid. Nitrogen number: 0.6 mmoles/g

### Example 35

### Preparation of a Matrix (M025)

Matrix (36.7 g) from example 14 (M004) was dissolved in boiling isopropanol (approximately 500 mL). Dimethyl sulfate (6.1 g, 48 mmol) was added dropwise. Stirring was continued for 30 min, at which point, the solvent was evaporated in vacuo. The resulting product was a light brown solid. Nitrogen number: 1.16 mmoles/g

### Example 36

### Preparation of a Matrix (M026)

Matrix (50 g) from example 33 (M0023) was dissolved in boiling isopropanol (approximately 300 mL). Dimethyl sulfate (6.24 g, 49.5 mmol) was added dropwise. Stirring was continued for 30 min at which point, the solvent was evaporated in vacuo. The resulting product was a light brown paste. Nitrogen number: 1.98 mmoles/g

### Example 37

### Preparation of a Matrix (M027)

Pentaethylenehexamine (46.5 g, 0.2 moles) was mixed with oleic acid (112.8 g; 0.4 moles). Sodium hypophosphite (0.5 g) was added. The mixture was kept at 135° C. for 1 hour and at 175° C. for an additional 5 h until no more water is formed. Heating was continued at 0.1 mbar for another 2 h. To 70 g of the reaction product, of phenyl isocyanate (20 g) was added at room temperature. The mixture was kept at 50° C. for 30 min. Finally the light brown product was dissolved in 500 ml isopropanol and 26 g dimethylsulfate were slowly added. The solution was boiled under reflux for 30 min, then the solvent was evaporated. Brown, honey-like oil.

### Example 38

### Preparation of a Matrix (M028)

Spermine (3.9 g, 19.2 mmol) and stearic acid (10.95 g, 38.5 mmol) were mixed together. Sodium hypophosphite (0.1 g) was added. The mixture was heated to 135° C. for 1 h and at 175° C. for an additional 2 h until water formation ceased. The mixture was heated to 175° C. for an additional 10 h at reduced pressure (0.1 mbar).

Nitrogen number: 2.07 mmol/g

### Example 39

### Preparation of a Matrix (M029)

3,3′-Diamino-N-methyldipropylamine (29 g, 0.2 moles) and behenic acid (135 g, 0.4 moles) were mixed together. Sodium hypophosphite (0.5 g) was added. The mixture was heated to 135° C. for 1 h and at 175° C. for an additional 2 h until water formation ceased. The mixture was kept at 175° C. for an additional 10 h at reduced pressure (0.1 mbar).

Nitrogen number: 1.28 mmol/g

### Example 40

### Preparation of a Matrix (M030)

Tetraethylenepentamine (28.4 g, 0.15 moles) is mixed with stearic acid (127.8 g, 0.45 moles). 0.5 g Sodium hypophosphite is added. The mixture is kept at 135° C. for 1 hour and at 175° C. for an additional 5 h until no more water is formed. Heating is continued for another 5 h at 0.1 mbar. Waxy, yellow solid.

Nitrogen number: 1.92 mmoles/g Tertiary Nitrogen: 1.89 mmoles/g

### Example 41

### Preparation of a Slow Release Formulation of Sulfentrazone with a Fatty Amine (F011)

Stearylamine (6500 mg) and sulfentrazone (3900 mg) were dissolved in approximately 150 mL of ethanol by slight heating. To this mixture was added 4000 AL of a 20% solution of polyacrylic acid in ethanol (800 mg). A flocculant precipitation occurs. The reaction mixture is dried in an open vessel at 50° C. for 6 h and left to harden at room temperature for 3 days. The resulting slightly brittle white material is milled to a particle size of less then 100 μm and then be applied as such.

### Example 42

### Formulation (F012)

Matrix (example 23, M013, 2 g) and mesotrione (0.85 g) were dissolved together in water (20 mL) at 50° C. The end product was a viscous, yellow emulsion. AI: mesotrione 30%.

### Example 93

### Formulation (F013)

Matrix (example 24, M014, 15 g) was finely ground and dissolved in hot ethanol (approximately 200 mL). To this solution was added mesotrione (6.78 g). Upon dissolution, stirring is continued without heating. The product precipitates as faint yellow powder. AI: 28.9%

### Example 44

### Formulation (F014)

Matrix (example 39, M029, 1.6 g) was finely ground and dissolved in hot ethanol (˜20 ml). To this solution mesotrione (0.7 g) was added. Upon dissolution, stirring was continued without heating. The product precipitates as faint yellow powder. AI: 30%.

### Example 45

### Formulation (F015)

Matrix (example 14, M014, 2.0 g) and 2,4-D (0.6 g) were heated until a homogeneous melt was formed. The solid product was ground to a powder with a particle size <100 μm. White powder. AI: 23%

### Example 46

### Formulation (F016)

Matrix (example 23, M013, 2 g) and 2,4-D (0.6 g) were dissolved with stirring in 20 ml of hot water. Once an emulsion has formed, the mixture was cooled down in an ice bath. Stable, white emulsion. AI: 2.65%

### Example 47

### Formulation (F017)

Matrix (example 23, M013, 2 g) and 2,4-D (1.2 g) were dissolved with stirring in 20 ml of hot water. Once an emulsion has formed, the mixture was cooled down in an ice bath. Stable, white emulsion. AI: 5.1%

### Example 48

### Formulation (F018)

Matrix (example 23, M013, 2 g) and dicamba (0.6 g) were dissolved with stirring in 20 ml of hot water. Once an emulsion has formed the mixture is cooled down in an ice bath. Stable, yellow micro-emulsion. AI: 2.65%

### Example 49

### Formulation (F019)

Matrix (example 23, M013, 2 g) and dicamba (1.2 g) were dissolved with stirring in 20 ml of hot water. Once an emulsion has formed, the mixture was cooled down in an ice bath. Stable, yellow micro-emulsion. AI: 5.1%

### Example 50

### Formulation (F020)

Matrix (example 40, M030, 2 g) and dicamba (0.8 g) were heated until a homogeneous melt was formed. The solid product was ground to a powder AI: 28%

### Example 51

### Formulation (F021)

Matrix (example 24, M014, 8.6 g) and dicamba (2.21 g) were heated until a homogeneous melt was formed. The solid product was ground to a powder with a particle size <100 μm. White powder. AI: 20%

### Example 52

### Formulation (F022)

Matrix (example 39, M029, 7.89 g) and dicamba (2.21 g) were heated until a homogeneous melt was formed. The solid product was ground to a powder with a particle size <100 μm. White powder. AI: 21.8%

### Example 53

### Formulation (F023)

Matrix (example 20, M010, 4.65 g), paraffin (5 g), mp<50° C., and dicamba (5 g) were mixed and heated until a homogeneous melt was formed. The solid product was ground to a powder with a particle size <100 μm. This powder was mixed thoroughly with 10.35 g talcum. White powder. AI: 20%.

### Example 54

### Formulation (F024)

Matrix (example 35, M025, 8.6 g) and dicamba (2.21 g) were mixed and heated until a homogeneous melt was formed. The solid product was ground to a powder with a particle size <<100 μm. Self-emulsifying brown powder. AI: 20%.

### Example 55

### Formulation (F025)

Matrix (example 23, M013, 6 g) and dicamba (3.6 g) were mixed and heated until dissolved. Triethyleneglycol (9.6 g) was added. Self-emulsifying brown oil. AI: 18.7%.

### Example 56

### Formulation (F026)

Matrix (example 29, M019, 8 g) and sulfentrazone (2.5 g) were heated until a homogeneous melt was formed. The solid product was ground to a powder with a particle size <100 μm. Yellow powder. AI: 23.8%

### Example 57

### Formulation (F027)

Matrix (example 39, M029, 6 g) and sulfentrazone (1.6 g) were heated until a homogeneous melt was formed. The solid product was ground to a powder with a particle size <100 μm. Yellow powder. AI: 23.8%

### Example 58

### Formulation (F028)

Matrix (example 39, M029, 2 g) and bromacil (0.66 g) were heated until a homogeneous melt was formed. The solid product was ground to a powder with a particle size <100 μm. Yellow powder. AI: 24.8%

### Example 59

### Formulation (F029)

Matrix (example 29, M019, 1.5 g) and bromacil (0.5 g) were heated until a homogeneous melt was formed. The solid product was ground to a powder with a particle size <100 μm. Yellow powder. AI: 25%

### Example 60

### Formulation (F030)

Matrix (example 20, M010, 10 g), ozokerit (10 g) and bromacil (10 g) were heated until a homogeneous melt was formed. The solid product was ground to a powder with a particle size <100 μm. This powder was mixed thoroughly with 6 g of ground talcum. White powder. AI: 19.5%.

### Example 61

### Formulation (F031)

Matrix (example 20, M010, 5.2 g) and paraffin (5.2 g) mp. <50° C., were heated until molten. Bromacil (3.46 g) was added. The mixture was heated until a homogeneous melt was formed. The solid product was ground to a powder with a particle size <100 μm. This powder was mixed thoroughly with 3.5 g of ground talcum. White powder. AI: 19%.

### Example 62

### Formulation (F032)

Matrix (example 20, M010, 5.0 g) and dammar (5.0 g) were mixed and heated until molten. Bromacil (5.0 g) was added. The mixture was heated until a homogeneous melt was formed. The solid product was ground to a powder with a particle size <100 μm. This powder was mixed thoroughly with 5.0 g of ground talcum. White powder. AI: 25%.

### Example 63

### Formulation (F033)

The mixture from example 62 was thoroughly mixed with powdered Bromacil (8 g). White powder. AI: 33%.

### Example 64

### Formulation (F034)

Matrix (example 28, M018, 9.0 g) and bromacil (6.0 g) were mixed and heated until a homogeneous melt was formed. The solid product was ground to a powder with a particle size <100 μm. This powder was mixed thoroughly with 1.5 g of ground talcum. White powder. AI: 36%.

### Example 65

### Formulation (F035)

Matrix (example 38, M028, 2 g) and imazapyr (1 g) were mixed and heated until a homogeneous melt was formed. The solid product was ground to a powder with a particle size <100 μm. Self-emulsifying white powder. AI: 33%.

### Example 66

### Formulation (F036)

Matrix (example 39, M029, 2 g) and imazapyr (0.66 g) were mixed and heated until a homogeneous melt was formed. The solid product was ground to a powder with a particle size <100 μm. Self-emulsifying white powder. AI: 25%.

### Example 67

### Formulation (F037)

Matrix (example 26, M016, 3 g), imazapyr (1.4 g) and beeswax (2.0 g) were mixed and heated until a homogeneous melt of low viscosity was formed. The solid product was formulated as granules. Light brown granules. AI: 22%

### Example 68

### Formulation (F038)

Matrix (example 24, M014, 3 g), imazapyr (1.4 g) and stearic acid (1.52 g) were mixed and heated until a homogeneous melt of low viscosity was formed. To this melt paraffin (1.35 g), mp<50° C., was added and stirred until homogeneous. The solid product was formulated as granules. Light brown granules. AI: 19%

### Example 69

### Formulation (F039)

Matrix (example 39, M029, 1.58 g) and Imazamox (0.66 g) were mixed and heated until a homogeneous melt was formed. The solid product was ground to a powder with a particle size <100 μm. Self-emulsifying white powder. AI: 25%.

### Example 70

### Formulation (F040)

Matrix (example 39, M029, 3.16 g) was molten in hot water (40.3 g). Imazamox (1.32 g) was added with vigorous stirring. The hot emulsion was cooled in an ice bath. White paste. AI: 2.9%

### Example 71

### Formulation (F041)

Matrix (example 24, M014, 2 g) and azoxystrobin (1.0 g) were mixed and heated until a homogeneous melt was formed. The solid product was ground to a powder with a particle size <100 μm. Hydrophobic white powder. AI: 33%.

### Example 72

### Formulation (F042)

Matrix (example 24, M014, 1.2 g) and azoxystrobin (0.2 g) were mixed and heated until a homogeneous melt was formed. The solid product was ground to a powder with a particle size <100 μm. This powder was mixed thoroughly with 0.3 g ground talcum. Emulsifiable white powder. AI: 11%.

### Example 73

### Formulation (F043)

Matrix (example 35, M025, 0.8 g) and azoxystrobin (0.2 g) were mixed and heated until a homogeneous melt was formed. The solid product was ground to a powder with a particle size <100 μm. Self-emulsifying white powder. AI. 20%.

### Example 74

### Formulation (F044)

Matrix (example 29, M019, 0.73 g) and azoxystrobin (0.25 g) were mixed and heated until a homogeneous melt was formed. The solid product was ground to a powder with a particle size <100 μm. Hydrophobic yellow powder. AI: 25.5%.

### Example 75

### Formulation (F045)

Matrix (example 26, M016, 2 g) and azoxystrobin (1.0 g) were mixed and heated until a homogeneous melt was formed. The solid product was ground to a powder with a particle size <100 μm. This powder was mixed thoroughly with 1 g of ground pumice. Emulsifiable white powder. AI: 25%.

### Example 76

### Formulation (F046)

Matrix (example 24, M014, 2 g) and glyphosate (0.46 g) were mixed and heated until a homogeneous melt was formed. The solid product was ground to a powder with a particle size <100 μm. Hydrophobic white powder. AI: 18.7%.

### Example 77

### Formulation (F047)

Matrix (example 39, M029, 1.9 g), sulfomethuron (0.1 g) and beeswax (2.0 g) were mixed and heated until a homogeneous melt of low viscosity ws formed. The solid product was to formulated as granules. Light yellow granules. AI: 2.5%

### Example 78

### Formulation (F048)

Matrix (example 20, M010, 1 g) and colophony (1.0 g) were heated until molten. Terbuthylazin (0.5 g) was added and heated until a homogeneous melt was formed. The solid product was ground to a powder with a particle size <100 μm. This powder was mixed is thoroughly with 0.5 g of ground talcum. Emulsifiable yellow powder. AI: 16%.

### Example 79

### Formulation (F049)

Matrix (example 30, M029, 1 g) and Dammar (0.5 g) were mixed and heated until a homogeneous melt was formed. Terbuthylazin (0.5 g) was added. Finally, ozokerit (0.5 g) was added and heated until a homogeneous melt was formed. The solid product was ground to a powder with a particle size <100 μm. Hydrophobic yellow powder. AI: 20%.

### Example 80

### Formulation (F050)

Matrix (example 35, M025, 2 g) and chlorothalonil (0.5 g) were mixed and heated until a homogeneous melt was formed. The solid product was ground to a powder with a particle size <100 μm. Dispersible powder. AI: 20%.

### Example 81

### Formulation (F051)

Matrix (example 13, M003, 5.0 g) and sulfentrazone (2.13 g) were mixed and heated until a homogeneous melt was formed. The solid product was ground to a powder with a particle size <100 μm. Dispersible light brown powder. AI: 30%

### Example 82

### Formulation of Metribuzine in a Fluorosilicate (F052)

Metribuzine (1.0 g) was dissolved in ethanol (15 mL). Water (5 mL) was added. Hydrofluoric acid (200 μl of a 50% solution of in water) was added with stirring. Tetraethylsilicate (7.62 mL) was added, and the mixture was heated until it starts gelling. Heating was continued overnight at 50° C. in an open vessel to dry and age the material. The resulting product was ground to a fine powder.

### Example 83

### Formulation of Metribuzine in a Polyethyleneimine-Fatty Acid Condensate (F053)

a) Lugalvan G35 (BASF) (70.4 g), behenic acid (17.6 g), and boric acid (500 mg) were combined and heated to 175° C. internal temperature with stirring and under a gentle stream of argon. After 3 h of heating, additional 52.8 g of behenic acid was added, and heating was continued for 2 additional hours.
b) 750 mg of a), and metribuzine (250 mg) were dissolved in warm ethyl acetate, and CO2 was bubbled through the solution until it was dry. The product was ground to a fine powder.

### Example 84

### Formulation of Metribuzine in a Polyethyleneimine Carbonate (F054)

Lupasol G100 (BASF) was dried to a residual water content of ca. 5%. A 12% solution of this material in 2-propanol was prepared and centrifuged. 5 mL of the supernatant and 200 mg of metribuzine were mixed, and CO2 was bubbled through, until the mixture was dry.

### Example 85

### Formulation of Metribuzine in a Borosilicate (F055)

Metribuzine (680 mg) and boric acid (395 mg) were dissolved in 9 mL methanol, and 4.25 mL of tetraethylsilicate was added. Water (2.5 mL) was added, and the mixture was shaken, until the initially-occurring turbidity was cleared. The mixture was kept overnight at 37° C. and air dried.

### Example 86

### Formulation of Sulfentrazone with a Fatty Amine (F056)

Stearylamine (3.23 g) and sulfentrazone (3.2 g) were dissolved in warm ethanol. A solution of non crosslinked polyacrylic acid (230 mg) dissolved in 10 mL of ethanol was added. The cloudy mixture was air-dried until a hard, malleable, white residue remains.

### Example 87

### Formulation of Herbicides with an N-alkyl-aminoglucit (F057)

a) Glucose (17 g) and stearylamine (32 g) were suspended in 500 mL methanol and stirred overnight. The solids were filtered off and dried to yield 41 g.
b) the product of a) was suspended in 500 mL of acetic acid and sodium borohydride (5.8 g) was added in portions. When the addition was complete, the solution was stirred for 15 min, and 50 mL acetone was added. All volatiles were distilled off with vacuum. The residual material was dissolved with warm ethyl acetate, and cyclohexane was added. Upon standing overnight, a precipitate was formed, that was discarded. The solution was treated with aqueous base. The resulting slurry was separated and the solid phase was washed with water to neutrality. This was dissolved in hot water, and the gel, that is formed upon cooling was dried by lyophilization.
c) 200 mg of b) was dissolved in 10 mL of boiling water, and the herbicide, dissolved in a suitable solvent, was added. The mixture was boiled, until all organics were evaporated, and cooled with vigourous stirring. After air drying, a solid product remains. Table 2 shows an illustrative but in no means limiting formulation examples:

TABLE 2
Different Formulations for Example 87

| | Active Ingredient | Amount (AI) | Amount Matrix b) |
|---|---|---|---|
| Entry | (AI) | [mg] | [mg] |
| F057a | Sulfentrazone | 205 mg | THF |
| F057b | Bromacil | 196 mg | THF |
| F057c | Quinclorac | 207 mg | Methanol |
| F057d | Terbuthylazine | 205 mg | THF |

### Example 88

### Formulation of Sulfentrazone in a Polyethyleneimine Phthalate (F058)

a) Polyethyleneimine (50% in water, 1.5 g PEI, 3 g) was dried (35 mbar, 100° C.), and dissolved in 20 mL of methanol. Powdered phthalic anhydride (2.8 g) was suspended in 20 mL of THF and added batch wise with heavy agitation to the PEI. After evaporation of all volatiles, the residue was heated to 180° C. for 3 h. Once cooled, the gel formed was broken to pieces in a mortar.
b) 178 mg of a) and sulfentrazone (90 mg) were combined with 400 μl of methanol, and vigorously shaken over night and air dried.

### Example 89

### Formulation (F059)

Matrix (example 20, M010, 5.0 g), dammar (2.5 g) and beeswax (2.5 g) were heated until molten. Bromacil (5.0 g) was added. The mixture was heated until a homogeneous melt was formed. The solid product was ground to a powder with a particle size <100 μm. This powder was mixed thoroughly with 3.0 g of ground talcum. White powder. AI: 27%.

### Example 90

### Formulation (F060)

Matrix (example 20, M010, 5.0 g) and beeswax (5.0 g) mp. were heated until molten. Bromacil (5.0 g) was added. The mixture was heated until a homogeneous melt was formed. The solid product was ground to a powder with a particle size <100 μm. This powder was mixed thoroughly with 3.0 g of ground talcum. White powder. AI: 27%.

### Example 91

### Formulation (F061)

Matrix (example 33, M023, 2.625 g) and Mesotrione (0.875 g) were heated until a homogeneous melt was formed. The solid product was ground to a powder with a particle size <100 μm. Self-emulsifying orange powder. AI: 21%

### Example 92

### Formulation (F062)

Matrix (example 28, M018, 2.0 g) and imidacloprid (0.221 g) were heated until a homogeneous melt was formed. The solid product was formulated as granules. Yellow granules. AI: 9.9%

### Example 93

### Formulation (F063)

The matrix of example 53a (120 mg) is dissolved together with imidacloprid (80 mg) in 3 ml of dichloromethane and 500 μl of methanol. CO₂ is bubbled through the solution, until all volatiles are evaporated and the mixture has reached room temperature. The whitish product is pistoned to achieve a powder. AI: 40%

### Example 94

### Formulation (F064)

a) Lupasol G10 (BASF) (19.7 g, 10 g PEI), behenic acid (20 g), and boric acid (250 mg) are combined and heated to an inner temperature of 170° C. under a gentle stream of Ar with stirring. After 90 min, the mixture is left cooling, and 3.3 g of 2-[2-(2-methoxyethoxy)ethoxy]acetic acid are added. The mixture is heated carefully, until a homogenous mixture is achieved, and then heated to 175° C. (internally) for 4 h. Then the reaction is left cooling, and dissolved in a boiling mixture of ethyl acetate and THF (1+1). Succinic acid anhydride (3.3 g), dissolved in hot ethyl acetate, is added and all volatiles are evaporated to yield a brown, brittle wax.
b) The product of a) (115 mg) and imidacloprid (80 mg) are combined in 3 ml of dichloromethane, and evaporated to dryness. The pale yellow product is pistoned to yield a powder.

TABLE 3
Summary of Formulation

| Code | Example | Matrix | Substance | Additive |
|---|---|---|---|---|
| F001 | 1 | PEI and stearic acid | Terbutylazine | — |
| F002 | 2 | PEI, Jeffamine ED 900, epichlorohydrin | Sulfentrazone | — |
| F003 | 3 | PEI and TEOS | Dicamba | — |
| F004 | 4 | PEI and TEOS | Sulfentrazone | — |
| F005 | 5 | Iron-(III)-Chloride | Mesotrion | — |
| F006 | 6 | Poly(diallyldimethylammonium chloride)/ | Glyphosate | — |
| | | TEOS | | |
| F007 | 7 | Polyacrylic acid/Stearylamine | Sulfentrazone | — |
| F008 | 8a-8n | PEI, Jeffamine ED 900, epichlorohydrin | Sulfentrazone, Dicamba, | — |
| | | | 2,4-D, or Quinclorac | |
| F009 | 9 | Matrix M018 | Clomazone | — |
| F010 | 10 | Triethoxy(octadecyl)silane/TEOS | Metribuzin | Colophony |
| F011 | 41 | Polyacrylic acid/Stearylamine | Sulfentrazone | |
| F012 | 42 | M013 | Mesotrione | — |
| F013 | 43 | M014 | Mesotrione | — |
| F014 | 44 | M029 | Mesotrione | — |
| F015 | 45 | M014 | 2,4-D | — |
| F016 | 46 | M013 | 2,4-D | — |
| F017 | 47 | M013 | 2,4-D | — |
| F018 | 48 | M013 | Dicamba | — |
| F019 | 49 | M013 | Dicamba | — |
| F020 | 50 | M030 | Dicamba | — |
| F021 | 51 | M014 | Dicamba | — |
| F022 | 52 | M029 | Dicamba | — |
| F023 | 53 | M010 | Dicamba | Paraffin |
| F024 | 54 | M025 | Dicamba | — |
| F025 | 55 | M013 | Dicamba | Triethylene gycol |
| F026 | 56 | M019 | Sulfentrazone | — |
| F027 | 57 | M029 | Sulfentrazone | — |
| F028 | 58 | M029 | Bromacil | — |
| F029 | 59 | M019 | Bromacil | — |
| F030 | 60 | M010 | Bromacil | Ozokerit |
| F031 | 61 | M010 | Bromacil | Paraffin, Talcum |
| F032 | 62 | M010 | Bromacil | Dammar, Talcum |
| F033 | 63 | F032 | Bromacil | — |
| F034 | 64 | M018 | Bromacil | Talcum |
| F035 | 65 | M028 | Imazapyr | — |
| F036 | 66 | M029 | Imazapyr | — |
| F037 | 67 | M016 | Imazapyr | Beeswax |
| F038 | 68 | M014 | Imazapyr | Paraffin |
| F039 | 69 | M029 | Imazamox | — |
| F040 | 70 | M029 | Imazamox | — |
| F041 | 71 | M014 | Azoxystrobin | — |
| F042 | 72 | M014 | Azoxystrobin | Talcum |
| F043 | 73 | M025 | Azoxystrobin | — |
| F044 | 74 | M019 | Azoxystrobin | — |
| F045 | 75 | M016 | Azoxystrobin | Pumice |
| F046 | 76 | M014 | Glyphosate | — |
| F047 | 77 | M029 | Sulfomethuron | — |
| F048 | 78 | M010 | Terbuthylazin | Talcum |
| F049 | 79 | M029 | Terbuthylazin | Dammar |
| F050 | 80 | M025 | Chlorothalonil | — |
| F051 | 81 | M003 | Sulfentrazone | — |
| F052 | 82 | SiO₂ | Metribuzine | HF |
| F053 | 83 | PEI/behenic acid | Metribuzine | — |
| F054 | 84 | PEI | Metribuzine | CO₂ |
| F055 | 85 | SiO₂ | Metribuzine | Boric acid |
| F056 | 86 | Stearylamine/Polyacrylic acid | Sulfentrazone | — |
| F057 | 87 | 6-Octadecylamino-hexane-1,2,3,4,5-pentaol | See Table 2 | — |
| F058 | 88 | Polyethyleneimine/Phthalic acid | Sulfentrazone | — |
| F059 | 89 | M010 | Bromacil | Dammar, Beeswax, |
| | | | | Talcum |
| F060 | 90 | M010 | Bromacil | Beeswax, Talcum |
| F061 | 91 | M023 | Mesotrione | — |
| F062 | 92 | M018 | Imidacloprid | — |
| F063 | 93 | PEI/behenic acid | Imidacloprid | CO₂ |
| F064 | 94 | PEI/behenic acid/2-[2-(2- | Imidacloprid | — |
| | | Methoxyethoxy)ethoxy]acetic | | |
| | | acid/succinic acid | | |
| F065 | 102 | M018 | Acetamiprid | |
| F066 | 103 | M027 | Acetamiprid | N-methylpyrrolidone |
| F067 | 103 | M014 | Epoxyconazole | Leunopan |
| F068 | 105 | M032 | Dicamba | Triethyleneglycol |
| F069 | 106 | M032 | Acetamiprid | Beeswax |
| F070 | 107 | M031 | Acetamiprid | Beeswax |
| F071 | 108 | MOM | Sulfentrazone | Polyacylic acid |
| F072 | 109 | M024 | Sulfentrazone | Polyacylic acid |
| F073 a-e | 110 | See Table 19 | — | — |

TABLE 4
Summary of Matrices

| | | Matrix | |
|---|---|---|---|
| Code | Example | Backbone | Modifications |
| | 1a | Polyethyleneimine | Stearic acid |
| | 2b | Polyethyleneimine | Jeffamine ED 900 (Huntsman), Epichlorohydrine |
| | 3 | Polyethyleneimine | SiO₂ |
| | 4 | SiO₂ | Polyethyleneimine |
| | 6 | Poly(diallyldimethyl)amine | SiO₂ |
| | 7 | Polyacrylic acid | Stearylamine |
| | 10 | SiO₂ | C18H37Si(O—)₃, Colophony |
| M001 | 11 | Pentaethylenehexamine | Stearic acid |
| M002 | 12 | Pentaethylenehexamine | Stearic acid and Oleic acid |
| M003 | 13 | Pentaethylenehexamine | Stearic acid |
| M004 | 14 | Pentaethylenehexamine | Stearic acid and Oleic acid |
| M005 | 15 | Tetraethylenepentamine | Stearic acid and Oleic acid |
| M006 | 16 | Pentaethylenehexamine | Tetradecanoic acid and Oleic acid |
| M007 | 17 | Triethylenetetramine | 2-ethylhexanoic acid and tetradecanoic acid |
| M008 | 18 | Tetraethylenepentamine | Stearic acid |
| M009 | 19 | Triethylenetetramine | Stearic acid |
| M010 | 20 | N-(2-Hydroxyethylenediamine) | Stearic acid |
| M011 | 21 | N-(2-Hydroxyethylenediamine) | Stearic acid and Oleic acid |
| M012 | 22 | N-(2-Hydroxyethylenediamine) | Stearic acid |
| M013 | 23 | Tetraethylenepentamine | Stearic acid and Oleic acid |
| M014 | 24 | 1,4-Bis(3-aminopropyl)piperazine | Stearic acid |
| M015 | 25 | 1,4-Bis(3-aminopropyl)piperazine | Stearic acid and Oleic acid |
| M016 | 26 | 1,4-Bis(3-aminopropyl)piperazine | Oleic acid |
| M017 | 27 | Tetraethylenepentamine | Stearic acid |
| M018 | 28 | M008 | 4-Chlorophenyl isocyanate |
| M019 | 29 | M008 | 4-Chlorophenyl isocyanate |
| M020 | 30 | M002 | Hexamethylene diisocyanate |
| M021 | 31 | M004 | Hexamethylene diisocyanate |
| M022 | 32 | M008 | Hexamethylene diisocyanate |
| M023 | 33 | M008 | Acetic anhydride |
| M024 | 34 | M009 | Methyl iodide |
| M025 | 35 | M004 | Dimethyl sulfate |
| M026 | 36 | M022 | Dimethyl sulfate |
| M027 | 37 | Pentaethylenehexamin | Oleic acid and phenyl isocyanate |
| | | | and dimethyl sulfate |
| M028 | 38 | Spermine | Stearic acid |
| M029 | 39 | 3,3′-Diamino-N-methyldiisopropylamine | Behenic acid |
| M030 | 40 | Tetraethylenepentamine | Stearic acid |
| | 83a | Polyethyleneimine (Lugalvan G35, BASF) | Behenic acid |
| | 88a | Polyethyleneimine | Phthalic acid |
| | 94a | Polyethyleneimine (Lupasol G10) | Behenic acid, 2-[2-(2-ethoxyethoxy)ethoxy]acetic |
| | | | acid, uccinic acid |
| M031 | 99 | Triethylenetetramine | Behenic Acid/MDI |
| M032 | 100 | Triethylenetetramine | Behenic Acid/Phenylisocyanate |
| M033 | 101 | Tetraethylenepentamine | Oleic Acid |
| M034 | 112 | 1,4-Bis(3-aminopropyl)piperazine | Behenic acid |

### Example 95

### Assay for Modification of Leaching Properties

### General Procedure

An 5 ml polyethylene syringe (cross sectional area approx. 1.3 cm2) without plunger was used as a column. A piece of round filter paper having the same area was placed at the bottom of the syringe to retain fine particles. An amount of soil or soil-sand mixture was placed in the syringe to a total volume of 4 mL. The soil was sieved (<0.5 mm particle size) prior to use to ensure uniform flow properties. Where soils have an excessive organic matter content, sand was added up to 50% of the total mass to improve water flow.

Prior to use, the columns were wetted with deionized water and allowed to drain until no further water leaves the column. A suspension, emulsion, or solution of the sample was prepared in deionized water, and 100 μl of this preparation, containing 200 μg of A.I. were applied on top of the column. Elution was achieved by addition of successive aliquots of 1 mL of deionized water. After each aliquot, the column was allowed to drain freely and the resulting eluate collected as fractions of approximately 1 mL. Each fraction was collected in a 1.5-mL centrifuge tube and to this 0.3 mL of methanol was added. The solution was mixed thoroughly, and centrifuged at 14,000×g for 5 minutes. The top 0.3 mL was transferred to an HPLC sample vial. The A.I. in the fractions was quantified by HPLC-MS-MS: an ionics EP10+ triple quadrupole mass spectrometer was tuned to efficiently detect the analytes via MS-MS and specific non-interfering molecular and daughter ions were assigned for each analyte. The instrument was calibrated using a mixed standard dilutions from 100 μM to 10 nM.

The analytes were separated using a 50×2.5 mm reprosil C18 column (Dr. Maisch GmbH, Ammerbuch, Germany) using an isocratic elution at 75% methanol in water containing 0.1% formic acid.

Data reported here were for various formulations of a given active ingredient. The graphs recorded the detector response for each fraction from a given column. Each data set in the graph was from a separate column eluted in parallel under the same conditions.

In certain instances, the elution time of the A.I. main peak was indicated by expressing the data as the % of total material eluted.

### Example 96

### Assay on Slow Release Activity

### General Procedure

A mixture of sieved (0.5 mm) soil and sand was prepared. 50 mL were filled into a pot with an area of 20 cm² (5 cm diameter) and wetted with deionized water. A slurry or solution of the formulation in deionized water was applied on top of the pot. Rainfall was simulated by repeated addition of water in 20-mL portions, each portion representing approximately 10 mm/m² of rain. 0.15 mL of rape seed (Brassica napus) were applied to the soil surface and covered with a thin layer of sand. (Any other seed of a plant susceptible to the herbicides tested may be used alternatively.) After 10 to 20 days, the seedlings were scored by number, height, appearance (color, leaf area), and, if harvested, by weight. Scores: 0=no effect; 1=25% suppression; 2=50% suppression; 3=75% suppression; 4=100% suppression

Score tables:

TABLE 5
Pre-emergent, canola, 3 kg/ha 2,4-D, 84 mm simulated
rainfall, assessed 14 days after planting

| | |
|---|---|
| Untreated | 0 |
| Commercial | 2 |
| F008i | 4 |
| F008j | 4 |
| F008k | 4 |

TABLE 6
Pre-emergent, canola, 3 kg/ha dicamba,
assessed 7 days after planting

| Rainfall [mm] | 14 | 28 | 42 | 84 |
|---|---|---|---|---|
| Untreated | 0 | 0 | 0 | 0 |
| Commercial formulation | 4 | 4 | 4 | 1 |
| F021 | 4 | 4 | 4 | 4 |

TABLE 7
Pre-emergent, canola, dicamba, 80 mm simulated
rainfall, assessed 7 days after planting

| formulation | amount [kg/ha] | score |
|---|---|---|
| Untreated | | 0 |
| Commercial formulation | 1 | 1 |
| Commercial formulation | 3 | 1 |
| F021 | 1 | 2 |
| F021 | 3 | 3 |

TABLE 8
Pre-emergent, canola, 3 kg/ha dicamba, 66 mm simulated
rainfall, assessed 14 days after planting

| | |
|---|---|
| Untreated | 0 |
| Commercial formulation | 1 |
| F008e | 3 |
| F008g | 3 |
| F008h | 3 |

TABLE 9
Pre-emergent, canola, 3 kg/ha bromacil, 120 mm simulated
rainfall, assessed 14 days after planting

| | |
|---|---|
| Untreated | 0 |
| Commercial formulation | 1 |
| F030 | 2 |
| F031 | 2 |
| F032 | 2 |
| F033 | 3 |
| F034 | 2 |

TABLE 10
Pre-emergent, canola, 400 g/ha sulfentrazone, 66 mm
simulated rainfall, assessed 14 days after planting

| | |
|---|---|
| Soil application, premergent to | 66 |
| canola | |
| Untreated | 0 |
| Commercial formulation | 3 |
| F007 | 4 |

TABLE 11
Pre-emergent, canola, 100 g/ha sulfentrazone, 84 mm
simulated rainfall, assessed 18 days after planting

| | |
|---|---|
| Untreated | 0 |
| Commercial formulation | 2 |
| F008b | 3 |
| F051 | 3 |
| F008c | 3 |

TABLE 12
Pre-emergent, canola, 300 g/ha sulfentrazone,
assessed 16 days after planting

| rainfall [mm] | 56 | 84 | 113 |
|---|---|---|---|
| Untreated | 0 | 0 | 0 |
| Commercial formulation | 4 | 3 | 2 |
| F026 | 3 | 3 | 3 |
| F027 | 4 | 3 | 4 |
| F008b | 3 | 2 | 2 |
| F056 | 3 | 3 | 2 |

TABLE 13
Pre-emergent, canola, 300 g/ha sulfentrazone,
assessed 16 days after planting

| rainfall [mm] | 56 | 84 | 113 |
|---|---|---|---|
| Untreated | 0 | 0 | 0 |
| Commercial formulation | 2 | 2 | 1 |
| F026 | 3 | 3 | 2 |
| F027 | 3 | 3 | 2 |
| F008b | 3 | 3 | 3 |
| F056 | 3 | 3 | 3 |

TABLE 14
Pre-emergent, canola, 3 kg/ha mesotrione, 200 mm simulated
rainfall, assessed 28 days after planting

| | |
|---|---|
| Untreated | 0 |
| Commercial formulation | 2 |
| F013 | 3 |
| F061 | 2 |

TABLE 15
Pre-emergent, canola, 500 g/ha imazapyr, 110 mm simulated
rainfall, assessment 28 days after planting

| | |
|---|---|
| Untreated | 0 |
| Commercial formulation | 3 |
| F035 | 4 |

### Example 97

### Assay on Foliar Uptake

Sunflowers or peas was seeded and left to germinate and emerge until the cotyledons were fully expanded, but the primary leaves have not appeared. The formulation was suspended in deionized water to achieve an established concentration (ca. 0.01 to 2% W/V), and an amount of this suspension (1 to 20 μL) is applied to one or two cotyledons of the seedlings. Within 5 to 30 days, an indicative parameter was observed, i.e. length of internodes for hormone type herbicides, or leaf damage for photosystem inhibitors or other signs of phytotoxicity for fungicides and insecticides. Other parameters include survival, plant height or fresh weight.

Scores: 0=no effect; 1=25% suppression; 2=50% suppression; 3=75% suppression; 4=100% suppression

Score tables:

TABLE 16
Sunflower, 20 μl, 0.06% based on dicamba

| days until assessment | 9 | 21 |
|---|---|---|
| Untreated | 0 | 0 |
| Commercial formulation | 2 | 3 |
| F021 | 2 | 2 |
| F025 | 3 | 4 |
| F024 | 2 | x |
| F008f | 1 | 1 |

TABLE 17
Sunflower, 10 μl, 0.075% based on sulfentrazone,
assessment 14 days after application

| | |
|---|---|
| Untreated | 0 |
| Commercial formulation | 4 |
| F027 | 2 |
| F008b | 1 |
| F056 | 1 |

TABLE 17
Sunflower, 10 μl, 0.075% based on sulfentrazone,
assessment 14 days after application

| | |
|---|---|
| Untreated | 0 |
| Commercial formulation | 4 |
| F057a | 1 |
| F058 | 2 |
| F008c | 2 |

### Example 98

### Rainfastness on Leaves

Established banana, rose, wheat or grape vine leaves were either obtained fresh from outdoor grown plants or from potted plants. When potted plants ere used, the leaf was treated attached to the plant. When outdoor plants were used, the leaf was cut under water and placed in water until used. Rose leaves were not kept longer than 8 h.

To determine rainfastness, a suspension of the test formulation was applied to the leaf surface with an application density of 20 μL/cm². If a drying time was foreseen, the formulation was allowed to dry for up to 4 hours. On the other hand, if a drying time was not foreseen, the leaf as immediately subject to a water stream as follows. Rainfastness is determined by resistance to a water stream. In this case, leaves were washed with a stream corresponding to greater than 100 mm/hour rainfall for ca. 120 s. Leaves were allowed to dry for 4 h and the treated areas are visually assessed both in normal and UV light, and then swabbed with alcohol containing swabs to remove active ingredient attached to the leaves. The treated areas of the leaf were then extracted using liquid phase extraction by grinding methanol 0.1% formic acid. Active ingredient in the leaf extracts or swabs was quantified by LC-MS-MS according to the method provided for assessing column eluates.

Scoring: 0=none visible; 1=25% visible; 2=50% visible; 3=75% visible; 4=100% visible

Score table:

TABLE 18
Banana, 20 μl, 300 μg/ml based on azoxystrobin,
>250 ml water immediately or after 2 h drying

| | | |
|---|---|---|
| Untreated | 0 | 0 |
| Commercial formulation | 0 | 0 |
| F042 | 1 | 1 |
| F044 | 1 | 2 |
| F041 | 1 | 1 |
| F043 | 1 | 3 |

### Example 99

### Preparation of a Matrix (M031)

Triethylenetetramine 60% (48.75 g, 0.2 mol) and behenic acid 85% (134.9 g, 0.4 mol) were mixed together. Sodium hypophosphite (0.5 g) was added. The mixture was heated to 135° C. for 1 h and at 175° C. for an additional 5 h until water formation ceased. The mixture was heated to 175° C. for an additional 10 h at reduced pressure (0.1 mbar). The resulting product was a yellow solid with a nitrogen number of 3.86 mmoles/g.

19.75 g of this condensate was melted and methylene-bis-(phenylisocyanate) (MDI, 10.0 g) were slowly added. A viscous orange melt was formed. The mixture was kept molten for another 15 min. Clear brittle orange solid.

### Example 100

### Preparation of a Matrix (M032)

Condensate (79 g) from example 99 (M031) was melted until water formation ceased. Phenylisocyanate (23.8 g) was slowly added. The mixture was kept molten for 15 min. The product obtained was a brittle orange solid.

### Example 101

### Preparation of a Matrix (M033)

Tetraethylenepentamine (47.2 g, 0.2 mol) and oleic acid (141 g, 0.5 mol) were mixed together. Sodium hypophosphite (0.5 g) was added. The mixture was heated to 135° C. for 1 h and at 175° C. for an additional 5 h until water formation ceased. The mixture was heated to 175° C. for an additional 8 h at reduced pressure (0.1 mbar). The resulting product was a light-brown oil. Nitrogen number: 3.61 mmoles/g

### Example 102

### Formulation (F065)

Matrix (example 28, M018, 10.4 g) and acetamiprid (2.6 g) were mixed and heated until a homogeneous melt was formed. The solid product was ground to a powder with a particle size <50 μm. Hydrophobic yellow powder. AI: 20%.

### Example 103

### Formulation (F066)

Matrix (example 37, M027, 8.5 g) and acetamprid (2.125 g) were mixed and dissolved in N-methylpyrrolidone (3.54 g).

Self-emulsifying brown oil.

AI: 15%.

### Example 104

### Formulation (F067)

Matrix (example 14, M014, 4.0 g) and matrix (example 35, M025, 2.0 g) and Leunapon F1618/25 (0.04 g) were mixed and heated until molten. Epoxiconazole was added and heated until a homogeneous melt was formed. The solid product was ground to a powder with a particle size <50 μm. Hydrophobic light-brown powder. AI: 24.9%.

### Example 105

### Formulation (F068)

Matrix (example 101, M033, 6.75 g) and dicamba (4.5 g) were mixed together with triethyleneglycol (3.75 g) and heated until a homogeneous mixture was formed.

Self-emulsifying brown oil. AI: 30%

### Example 106

### Formulation (F069)

Matrix (example 100, M032, 1.6 g), beeswax (0.5 g), and acetamiprid (0.4 g) were mixed and heated until a homogeneous melt was formed. The product was formulated as granules. Yellow granules. AI: 16%.

### Example 107

### Formulation (F070)

Matrix (example 99, M031, 3.0 g) and sulfentrazone (1.0 g) were mixed and heated until a homogeneous melt was formed. The solid product was ground to a powder with a particle size <50 μm. Hydrophobic powder. AI: 25%.

### Example 108

### Formulation (F071)

Matrix (example 24, M014, 10.0 g) and polyacrylic acid 1800 (0.5 g) are mixed and molten until homogeneous. Sulfentrazone (2.5 g) was stirred in and kept molten until homogeneous. The solid product was ground to a powder with a particle size <50 μm. Hydrophobic powder. AI: 25%. Light-brown powder.

### Example 109

### Formulation (F072)

Matrix (example 34, M024, 5.0 g) and polyacrylic acid 1800 (0.25 g) are mixed and molten until homogeneous. Sulfentrazone (1.25 g) was stirred in and kept molten until homogeneous. The solid product was ground to a powder with a particle size <50 μm. Hydrophobic yellow powder. AI: 25%.

### Example 110

### Formulation of Sulfentrazone with a Fatty Amine (F011) and a Polymeric Acid

Stearylamine (25 g) and sulfentrazone (15 g) were dissolved in approximately 300 mL of ethanol by gentle heating. To this mixture was added 15 mL of a 20% solution of polyacrylic acid (MW approx. 1800) in ethanol (3 g). A flocculant precipitation occurs. The reaction mixture is heated to approx. 65° C., until all precipitates are dissolved (if necessary, mechanical action is applied). It is dried in an open vessel with mechanical stirring at 65° C., until a viscous residue remains. This residue is dried in an open vessel at 70° C. over night to achieve a viscous, clear liquid. The liquid is cooled fast to room temperature and kept so for 2 days. The material is milled to sub-50 μm particle size.

### Example 111

### Formulation of Metribuzin (F073)

Procedure 1. Metribuzin and matrix were dissolved in ethanol and heated to 78° C. To the hot solution was added dropwise over 30 min a solution of poly(acrylic acid) (Typical Mw 1,800) in ethanol. The suspension was heated until a homogenous solution is formed. The solution is poured into a container with a large flat surface area and the ethanol allowed to evaporate. The resulting residue was dried in the oven (70° C.) and the resulting crystalline product was pulverized to a particle size of 50 μm or smaller.

Procedure 2. Metribuzin and the matrix were mixed and ground to a fine powder, transferred to a crystallizing dish and melted on a hot plate. Under agitation, hot ethanolic solution of poly(acrylic acid) (Typical Mw 1,800) was added dropwise to the melt. The resulting gel-like substance was allowed to cool slowly. The brittle and hard product was pulverized to a particle size of 50 μm or smaller.

TABLE 19
Formulation with Metribuzin

| | | | | | | Pro- |
|---|---|---|---|---|---|---|
| | | Amount | Amount | Amount | % | ce- |
| Entry | Matrix | Matrix | PAA | Metribuzin | Metribuzin | dure |
| F073a | M032 | 10.5 | 0.8 | 3.8 | 25 | 1 |
| F073b | M034 | 10.5 | 0.8 | 3.8 | 25 | 2 |
| F073c | Stearyl | 10.8 | 3.2 | 4.7 | 25 | 1 |
| | amine | | | | | |
| F073d | Stearyl | 10.8 | 3.2 | 4.7 | 25 | 1, 2 |
| | amine | | | | | |
| F073e | M034 | 9.2 | 1.5 | 3.3 | 24 | 2 |
| F073f | M032 | 10.5 | 0.8 | 3.8 | 25 | 1 |
| F073e | M032 | 10.5 | 1.6 | 3.8 | 24 | 2 |

### Example 112

### Preparation of a Matrix (M034)

1,4-Bis(3-aminopropyl)piperazine (40 g, mol) and behenic acid (135 g, mol) were mixed together. Sodiumhypophosphite (0.5 g) was added. The mixture was heated to 135° C. for 1 h and at 175° C. for an additional 5 h until water formation ceased. The mixture was heated to 175° C. for an additional 5 h at reduced pressure (0.1 mbar). The resulting product was a waxy, brown solid.

Nitrogen number: 2.54 mmoles/g

### Example 113

### Preparation of a Formulation of a Herbicide with a Fatty Amine

Stearylamine (1.55 g) and 2,4-D (1.42 g) are melted together at 75-80° C. until a homogenous solution is formed. After cooling over night, the resulting slightly brittle white material can be carefully milled to a particle size of less then 50 μm and then be applied as such. Alternatively, 2.5 g of stearylamine are melted and 2.4 g of sulfentrazone are dissolved in it. The homogenous clear melt cooled, ground and sieved to the desired particle size. Alternatively, it may be sprayed in the molten state in a cooled tower to render particles defined by spray temperature and carrier gas pressure.

### Example 114

### Preparation of a Formulation of 2,4-D with a Fatty Amine

Stearylamine (145 g) is melted, and 2,4-D (113.9 g) is dissolved, until a clear solution is formed. The material is poured on a plate of room temperature and left cooling, then grinded and sieved through a 50 μm sieve.

### Example 115

### Preparation of a Formulation of a Herbicide with a Fatty Amine

Noram 42® (3.44 g is melted, and 2,4-D (2.45 g) is dissolved, until a clear solution is formed. The material is poured on a plate of room temperature and left cooling, then grinded and sieved to the desired particle size. Alterantively, 10.23 g of Noram 42® are melted and 9.06 g of sulfentrazone is dissolved in it. The homogenous clear melt cooled, ground and sieved to the desired particle size. Alternatively, it may be sprayed in the molten state in a cooled tower to render particles defined by spray temperature and carrier gas pressure.

### Example 116

### Preparation of a Formulation of Metazachlor with Rosin

a) 99 g of colophonium (“rosin”) and 29.7 g of polyethylenimine (50% solution in water, 14.85 g of polyethylenimine) are mixed and heated with stirring to 195° C. under vacuum (1 mbar) for 90 min. After cooling to RT, a glass is formed, that can be used for the next step.
b) 1.62 g of the product of a), and 0.59 g of rosin are mixed and melted at 160° C. 1.06 g of metazachlor are added with heavy stirring, and as soon as everything is dissolved (ca. 3-4 min), the mixture is poured on a cool plate. The brittle glassy product is powdered and sieved to the required particle size.

Dissolution of the powder in 2-propanol and analysis by HPLC-UV shows a metazachlor content of 28%.

### Example 117

### Application of a Formulation as a Melted Concentrate

In certain instances it is desirable to apply a bioactive substance in a minimal volume. In such circumstances, a formulation such as that in examples 7, 45, 50 or 67 is formed into granules in the range of 2 mm in diameter that are loaded into a hopper. The hopper is connected to an air stream and the hopper distributes the granules to the air stream using a metered archemedes screw. The airstream forces the granules to a heated compartment where they melt and the molten material is atomized and sprayed in the air stream toward its target. Alternatively, the molten formulation is contacted to a heated spinning disk which uses centripetal force to create particles that leave the disk and which are then distributed to the target.

### INDUSTRIAL APPLICABILITY

The matrices and formulations based on these are suitable for use in agriculture, industrial pest control, and as vehicles and excipients for biologically active substances such as pharmaceuticals, cosmetics and personal care products.

### REFERENCES CITED

U.S. PATENT DOCUMENTs

| | | | |
|---|---|---|---|
| 20070149409 | June 2007 | Burnet, et.al. | 504/360; 424/410 |
| 6,096,686 | August 2000 | Gressel, et.al. | 504/100; 504/206 |
| 3,664,999 | May 1972 | Khusid et al. | 260/231 |
| 3,914,230 | October 1975 | Hyson | 449/755 |

We claim:

1. A formulation, comprising a mixture of:
a) a matrix, which may be defined as being either an monomeric, dimeric, multimeric, oligomeric- or a polymeric backbone which may be acidic or basic in nature, branched or linear, crosslinked or non-crosslinked and may exist in the free form or covalently linked to, mixed with and/or grafted with neutral components which includes but is not limited to polyethylene glycol, propylene glycol, or fatty acid condensates,
b) a bioactive substance imbedded or trapped within the matrix and constitutes at least 8% w/w of the formulation;
c) an additive or a modifier which may be 0 to 40% of the composition by weight.

2. A formulation, as in claim 1, wherein the matrix is of polymeric nature defined as having at least ten similar repeating units.

3. A formulation as in claim 2, wherein the polymeric backbone is of basic nature.

4. A formulation as in claim 3, wherein the polymeric backbone may be polyamines, which includes but not limited to: polyethylenimine, polypropylenimine, polyvinylamine or polypropylenamine.

5. A formulation as in claim 4, wherein polyamine is crosslinked with a bi- or polyfunctional agent which includes but are not limited to epichlorohydrin, di- or poly-epoxide like diglycidylglycerol or diglycidylethyleneglycol or 2-[[3-(oxiran-2-ylmethoxy)-2,2-bis(oxiran-2-ylmethoxymethyl)propoxy]methyl]-oxirane, di- or polycarboxylic acid like phthalic acid, succinic acid, diglycolic acid, adipic acid, di- or polyisocyanate like phenyl diisocyante or poly[(phenyl isocyanate)-co-formaldehyde].

6. A formulation as in claim 5, wherein the polymeric backbone is grafted with either polyethylene glycol, polypropylene glycol, polyvinyl alcohol or mixtures thereof.

7. A formulation as in claim 6, wherein the bioactive substance constitutes at least 8% w/w of the total mass.

8. A formulation as in claim 7, wherein the bioactive substance is an acidic compound having a pKa of 7 and below.

9. A formulation as in claim 2, wherein the polymeric backbone is of acidic nature.

10. A formulation as in claim 9, wherein the polymeric backbone is a polycarboxylic acid, which includes but not limited to polyacrylic acid or polymethacrylic acid.

11. A formulation as in claim 9, wherein the polymeric backbone is a polysiloxane of the type R1-(SiR2R3)-O—(SiR4[OH])—R5, or condensation products thereof, where R1,R2,R3,R4,R5 can be any substituent encompassing but is not limited to the following groups: H, OH, C1-C22-Alkyl, C1-C22-Alkoxy, C2-C22-alkenyl, aryl, aryloxy (alkylaryl, alkylaryloxy), alkoxyalkyl, alkoxyallyloxy heteroaryl, or heteroaryloxy.

12. A formulation as in claim 1, wherein an additive or modifier, includes but not limited to: Polyethylene glycol, polypropylene glycol, polyvinyl alcohol, polyacrylates, fatty acids with at least 6 carbon atoms, alkyl amines with at least 6 carbon atoms, biopolymers (for example, but not limited to cellulose, starch, chitin, chitosan, lignin, proteins), minerals (for example, but not limited to: vermiculite, kieselguhr, montmorillionite, talcum), resins (for example, but not limited to: dammar, olibanum, myrrh, galipot, rosin), paraffin or mixtures, combinations, condensates or copolymers thereof.

13. A formulation, comprising a mixture of:
1, A matrix which is composed of one or a combination of an alkylamine, an alkylacid, the reaction product of a carboxylic acid and/or a carboxylic anhydride and/or an ester and/or a fat with the primary or secondary amino functions of an amino compound and/or the hydroxyl functions of an amino alcohol;
2, A bioactive substance imbedded or trapped within the matrix;
3, One or more additives or modifiers which may be 0 to 95% of the composition by weight.

14. A matrix as in claim 13 where the molar ratio of carboxylic functions to amino compound is equal or higher than 1.

15. A matrix as in claim 13 where the carboxylic acid or -ester or -anhydride may be aliphatic or aromatic in nature, contains no less than six- but no more than 24 carbon atoms and may be saturated or unsaturated, straight-chain or branched and mixtures thereof.

16. A carboxylic acid as in claim 15 that may or may not contain hydroxyls and/or oligoethylene- or propylene oxides.

17. A matrix as in claim 13 where the amino compound is monomeric or oligomeric in nature and contains at least two primary or one primary and one secondary or two secondary amines.

18. A matrix as in claim 13 where the amino compound is monomeric or oligomeric in nature and contains at least one primary or secondary amine and one hydroxyl function.

19. A matrix as in claim 13 where the amino compound is monomeric or oligomeric in nature and contains at least two hydroxyl functions and one tertiary amine.

20. A matrix as in claim 13 wherein the matrix is cross-linked with a bi- or poly-functional electrophile which includes but are not limited to hexamethylene diisocyanate, epichlorohydrin, adipic acid, sebacic acid, trimellitic acid, pyromellitic acid, etc.

21. A matrix as in claim 13 that may be further modified by electrophiles which include but are not limited to acetic anhydride, phenyl isocyanate, chloroacetic acid and salts thereof, dimethyl sulfate, benzyl chloride, methyl chloride, etc.

22. A formulation as in claim 1, where the bioactive substance is a pharmaceutical agent.

23. A formulation as in claim 13, where the bioactive substance is a pharmaceutical agent.

24. A formulation as in claim 1, where the bioactive substance is a pesticide.

25. A formulation as in claim 24 in which the bioactive substance is a herbicide.

26. A formulation as in claim 24 in which the bioactive substance is a fungicide.

27. A formulation as in claim 24 in which the bioactive substance is an insecticide.

28. A formulation as in claim 24 in which the bioactive substance is a nematicide.

29. A formulation as in claim 13, where the bioactive substance is a pesticide.

30. A formulation as in claim 29 in which the bioactive substance is a herbicide.

31. A formulation as in claim 29 in which the bioactive substance is a fungicide.

32. A formulation as in claim 29 in which the bioactive substance is an insecticide.

33. A formulation as in claim 29 in which the bioactive substance is a nematicide.

34. A formulation as in claim 13, wherein an additive or modifier is selected from the following: Polyethylene glycol, polypropylene glycol, polyvinyl alcohol, polyacrylates, polyethylene imines, polyvinyl amines, fatty acids with at least 6 carbon atoms, alkyl amines with at least 6 carbon atoms, biopolymers (examples of which are, but not limited to cellulose, starch, chitin, chitosan, lignin, proteins), minerals (examples of which are, but not limited to: vermiculite, kieselguhr, montmorillionite, talcum), resins (examples of which are, but not limited to: dammar, colophony, olibanum (frankincense), myrrh, galipot, rosin), paraffin or mixtures, combinations, condensates or copolymers thereof and which constitutes 0 to 95% of the composition by weight.

35. A formulation as in claim 13, where the additive is an anionic, a neutral or a cationic surfactant or mixtures thereof and which constitutes 0 to 95% of the composition by weight.

36. A formulation as in claim 13 where the additive is a solvent.

37. A formulation as in claim 36 where the solvent is selected from methanol, ethanol, isopropanol, ethyleneglycol, water miscible solvents such as acetone, methylformamide, methylsulfoxide, N-methylpyrrolidone, water immiscible solvents such as hexane, heptane, toluene, naphta, and/or mixtures thereof and which constitutes 0 to 95% of the composition by weight.

38. A formulation according to claim 1 which is ground to an average particle size of less than 1 mm in diameter.

39. A formulation according to claim 13 which is ground to an average particle size of less than 1 mm in diameter.

40. A method of formulating a pesticide according to claim 38 in which the dried mixture is ground to an average particle size of less than 0.2 mm in diameter.

41. A method of formulating a pesticide according to claim 39 in which the dried mixture is ground to an average particle size of less than 0.2 mm in diameter.

42. A method of formulation wherein a bioactive substance is mixed with a matrix and/or additive and this mixture is melted in the course of preparation, processing or use

43. A method as in claim 42 wherein the formulation is melted as a means of incorporating the bioactive substance.

44. A method as in claim 42 wherein the formulation is melted and sprayed to form granules or particles which are then packaged ready for use.

45. A method as in claim 42 wherein the formulation is melted as a means of applying the bioactive substance to its target.
